(19) United States
(12) Patent Application Publication
(10) Pub. No.: US 2013/0324482 A1
(43) Pub. Date: Dec. 5, 2013

(54) COMPSTATIN ANALOGS FOR TREATMENT OF RHINOSINUSITIS AND NASAL POLYPOSIS

(71) Applicants: Cedric Francois, Louisville, KY (US); Pascal Deschatelets, Louisville, KY (US)

(72) Inventors: Cedric Francois, Louisville, KY (US); Pascal Deschatelets, Louisville, KY (US)

(73) Assignee: APELLIS PHARMACEUTICALS, INC., Crestwood, KY (US)

(21) Appl. No.: 13/809,274

(22) PCT Filed: Jul. 8, 2011
(86) PCT No.: PCT/US11/43466

§ 371 Date: Aug. 14, 2013

Related U.S. Application Data
(60) Provisional application No. 61/363,110, filed on Jul. 9, 2010.

(51) Int. Cl. C07K 7/54; A61K 45/06; A61K 38/12

(52) U.S. Cl. CPC C07K 7/54; A61K 38/12; A61K 45/06
USPC 514/21.1

(57) ABSTRACT

In some aspects, the present invention provides methods treating a subject in need of treatment for chronic rhinosinusitis or nasal polyposis, the methods comprising administering a complement inhibitor such as a compstatin analog to the subject. In some embodiments, the complement inhibitor is administered intranasally, e.g., in a nasal spray.

### CROSS-REFERENCE TO RELATED APPLICATIONS

This application claims the benefit of and priority to U.S. Provisional Patent Application No. 61/363,110, filed Jul. 9, 2010, the entire contents of which are incorporated herein by reference.

### BACKGROUND OF THE INVENTION

Chronic rhinosinusitis (CRS) is one of the most common chronic diseases, affecting an estimated 14%-16% of the US population. Common symptoms of CRS include nasal obstruction, blockage, or congestion; facial pain or pressure; nasal discharge, and reduction or loss of sense of smell. These troublesome symptoms can significantly impair patients' quality of life and result in significant utilization of health care resources. Therapy for CRS often includes oral antibiotics, topical corticosteroids, decongestants, and mucolytics. However, medical management alone is insufficient to relieve symptoms in a significant proportion of patients with CRS. Functional endoscopic sinus surgery (FESS) has become the treatment of choice for patients with medically refractory CRS. More than 200,000 FESS procedures per year are performed in the United States alone. Although most patients who undergo FESS for chronic sinusitis experience significant symptomatic relief, it has been reported that up to 23% of patients ultimately require revision surgery for continued or recurrent sinus symptoms after initial surgery, with a diminishing success rate.

Nasal polyps are abnormal growths that arise from the mucosa of the paranasal sinuses or the nasal cavity. The estimated prevalence of nasal polyposis is about 4% in the general population. The condition predominantly affects adults, usually individuals older than 20 years of age. Symptoms of nasal polyposis can vary depending on the size and location of the polyp(s). Nasal obstruction, rhinorrhea, and postnasal drip are common. Anosmia or hyposmia, with an ensuing alteration in taste, occur frequently. Nasal polyps can also produce sleep disturbances, snoring, headache, and facial pain. The mainstays of current medical therapy for small to moderate sized NPs are oral and/or topical corticosteroids. In the case of larger polyps or inadequate response to medical therapy, surgical polypectomy or endoscopic sinus surgery may be performed. Unfortunately, polyps frequently recur following surgery. Patients may be subjected to multiple surgeries with their attendant costs and risks of complications and/or may experience a long-term reduction in quality of life due to continued symptoms.

There is a significant need in the art for new therapies for chronic rhinosinusitis and nasal polyposis.

### SUMMARY OF THE INVENTION

The invention provides methods of treating a subject in need of treatment for chronic rhinosinusitis (CRS), the methods comprising administering a compstatin analog to the subject. In some embodiments, the subject suffers from CRS with nasal polyposis. In some embodiments, the subject suffers from asthma, non-steroidal anti-inflammatory (NSAID) sensitivity, and CRS with nasal polyposis. In some embodiments, the subject suffers from CRS without nasal polyposis. In some embodiments, the subject has CRS that is refractory to nasal and/or systemic (e.g., oral) corticosteroid therapy.

In another aspect, the invention provides methods of treating a subject in need of treatment for nasal polyposis, the methods comprising administering a compstatin analog to the subject. In some embodiments, the subject suffers from nasal polyposis and CRS. In some embodiments, the subject suffers from nasal polyposis and not CRS. In some embodiments, the subject suffers from asthma and nasal polyposis. In some embodiments, the subject suffers from NSAID sensitivity and nasal polyposis. In some embodiments, the subject suffers from asthma, NSAID sensitivity, and nasal polyposis. In some embodiments, the subject suffers from recurrent nasal polyps. In some embodiments, the subject suffers from bilateral nasal polyps. In some embodiments, the subject has CRS that is refractory to nasal and/or oral corticosteroid therapy.

In some embodiments of the methods of treatment of the invention, the compstatin analog is administered intranasally. In some embodiments, the compstatin analog is administered in a nasal spray.

All articles, books, patent applications, patents, other publications, and electronic databases mentioned in this application are incorporated herein by reference. In the event of a conflict between the specification and any of the incorporated references the specification (including any amendments thereto) shall control. Unless otherwise indicated, art-accepted meanings of terms and abbreviations are used herein.

### DEFINITIONS

The term “antibody” refers to an immunoglobulin or a derivative thereof containing an immunoglobulin domain capable of binding to an antigen. The antibody can be of any species, e.g., human, rodent, rabbit, goat, chicken, etc. The antibody may be a member of any immunoglobulin class, including any of the human classes: IgG, IgM, IgA, IgD, and IgE, or subclasses thereof such as IgG1, IgG2, IgG3, IgG4. In various embodiments of the invention the antibody is a fragment such as an Fab′, F(ab′)2, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. An antibody may be a chimeric antibody in which, for example, a variable domain of rodent origin or non-human primate origin is fused to a constant domain of human origin, or a “humanized” antibody in which some or all of the complementarity-determining region (CDR) amino acids that constitute an antigen binding site (sometimes along with one or more framework amino acids or regions) are “grafted” from a rodent antibody (e.g., murine antibody) or phage display antibody to a human antibody, thus retaining the specificity of the rodent or phage display antibody. Thus, humanized antibodies may be recombinant proteins in which only the antibody complementarity-determining regions are of non-human origin. It will be understood that “originate from or derived from” refers to the original source of the genetic information specifying an antibody sequence or a portion thereof, which may be different from the species in which an antibody is initially synthesized. For example, “human” domains may be generated in rodents whose genome incorporates human immunoglobulin genes. See, e.g., Vaughan, et al, (1998), Nature Biotechnology, 16: 535-539, e.g., to generate a fully human antibody. An antibody may be polyclonal or monoclonal, though for purposes of the present invention monoclonal antibodies are generally preferred as therapeutic agents. Methods for generating antibodies that specifically bind to virtually any molecule of interest are known in the art. For example, monoclonal or polyclonal antibodies can be purified from natural sources, e.g., from blood or ascites fluid of an animal that produces the antibody (e.g., following immunization with the molecule or an antigenic fragment thereof) or can be produced recombinantly, in cell culture and, e.g., purified from culture medium, in transgenic organisms, or at least in part using chemical synthesis. Affinity purification may be used, e.g., protein A/G affinity purification and/or affinity purification using the antigen as an affinity reagent. Suitable antibodies can be identified using phage display and related techniques. See, e.g., Kaser, M. and Howard, G., “Making and Using Antibodies: A Practical Handbook” CRC Press, 2006, and/or Sidhu, S., “Phage Display in Biotechnology and Drug Discovery”, CRC Press, Taylor and Francis Group, 2005, for further information. Methods for generating antibody fragments are well known. For example, F(ab′)₂ fragments can be generated, for example, through the use of an Immunopure F(ab′)₂ Preparation Kit (Pierce) in which the antibodies are digested using immobilized pepsin and purified over an immobilized Protein A column. The digestion conditions (such as temperature and duration) may be optimized by one of ordinary skill in the art to obtain a good yield of F(ab′)₂. The yield of F(ab′)₂ resulting from the digestion can be monitored by standard protein gel electrophoresis. F(ab′) can be obtained by papain digestion of antibodies, or by reducing the S—S bond in the F(ab′)₂. Antibodies known in the art as diabodies, minibodies, or nanobodies can be used in various embodiments. Bispecific or multispecific antibodies may be used in various embodiments. As used herein, a “single-chain Fv” or “scFv” antibody fragment comprises the V_{H} and V_{L} domains of an antibody, wherein these domains are present in a single polypeptide chain. Typically, a scFv antibody further comprises a polypeptide linker between the V_{H} and V_{L} domains, although other linkers could be used to connect the domains in certain embodiments.

The terms “approximately” or “about” in reference to a number generally include numbers that fall within ±10%, in some embodiments ±5%, in some embodiments ±1%, in some embodiments ±0.5% of the number unless otherwise stated or otherwise evident from the context (except where such number would impermissibly exceed 100% of a possible value).

A “complement component” or “complement protein” is a protein that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, and properdin. Components of the lectin pathway include, e.g., MBL2, MASP-1, and MASP-2. Complement components also include cell-bound receptors for soluble complement components, wherein such receptor mediates one or more biological activities of such soluble complement component following binding of the soluble complement component. Such receptors include, e.g., C5a receptor (C5aR), C3a receptor (C3aR), Complement Receptor 1 (CR1), Complement Receptor 2 (CR2), Complement Receptor 3 (CR3, also known as CD45), etc. It will be appreciated that the term “complement component” is not intended to include those molecules and molecular structures that serve as “triggers” for complement activation, e.g., antigen-antibody complexes, foreign structures found on microbial or artificial surfaces, etc.

A “complement regulatory protein” is a protein involved in regulating complement activity. A complement regulatory protein may down-regulate complement activity by, e.g., inhibiting complement activation or by inactivating or accelerating decay of one or more activated complement proteins. Examples of complement regulatory proteins include C1 inhibitor, C4 binding protein, clusterin, vitronectin, CFH, factor I, and the cell-bound proteins CD46, CD55, CD59, CR1, CR2, and CR3.

“Polypeptide”, as used herein, refers to a polymer of amino acids, optionally including one or more amino acid analogs. A protein is a molecule composed of one or more polypeptides. A peptide is a relatively short polypeptide, typically between about 2 and 60 amino acids in length, e.g., between 8 and 40 amino acids in length. The terms “protein”, “polypeptide”, and “peptide” may be used interchangeably. Polypeptides used herein may contain amino acids such as those that are naturally found in proteins, amino acids that are not naturally found in proteins, and/or amino acid analogs that are not amino acids. As used herein, an “analog” of an amino acid may be a different amino acid that structurally resembles the amino acid or a compound other than an amino acid that structurally resembles the amino acid. A large number of art-recognized analogs of the 20 amino acids commonly found in proteins (the “standard” amino acids) are known. One or more of the amino acids in a polypeptide may be modified, for example, by the addition of a chemical entity such as a carbohydrate group, a phosphate group, a farnesyl group, an isofarnesyl group, a fatty acid group, a linker for conjugation, functionalization, or other modification, etc. Certain non-limiting suitable analogs and modifications are described in WO2004026328. The polypeptide may be acetylated, e.g., at the N-terminus and/or amidated, e.g., at the C-terminus.

“Reactive functional groups” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those frequently used to prepare bioconjugates, e.g., N-hydroxysuccinimide esters, maleimides, sulfhydryls, and the like (see, for example, Hermanson, G., Bioconjugate Techniques, Academic press, San Diego, 1996). Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press. San Diego, 1989).

As used herein, a “subject” is typically a human or a non-human primate (e.g., a cynomolgus monkey, rhesus monkey, or baboon) or a non-primate animal that comprises a primate (e.g., human) C3. In some embodiments the subject is male. In some embodiments the subject is female. In some embodiments the subject is an adult, e.g., a human at least 18 years of age.

“Treating”, as used herein, refers to providing treatment, i.e, providing any type of medical or surgical management of a subject. The treatment can be provided in order to reverse, alleviate, inhibit the progression of, prevent or reduce the likelihood of a disease, disorder, or condition, e.g., CRS and/or nasal polyposis, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of CRS and/or nasal polyposis, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of this invention can be administered to a subject who has developed CRS and/or nasal polyposis or is at increased risk of developing CRS and/or nasal polyposis relative to a member of the general population. A composition of this invention can be administered prophylactically, i.e., before development of any symptom or manifestation of CRS and/or nasal polyposis. Typically in this case the subject will be at risk of developing the condition.

As used herein, “alkyl” refers to a saturated straight, branched, or cyclic hydrocarbon having from about 1 to about 22 carbon atoms (and all combinations and subcombinations of ranges and specific numbers of carbon atoms therein), with from about 1 to about 12, or about 1 to about 7 carbon atoms being preferred in certain embodiments of the invention. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2,2-dimethylbutyl, and 2,3-dimethylbutyl.

As used herein, “halo” refers to F, Cl, Br or I.

As used herein, “alkanoyl” refers to an optionally substituted straight or branched aliphatic acyclic residue having about 1 to 10 carbon atoms (and all combinations and subcombinations of ranges and specific number of carbon atoms) therein, e.g., from about 1 to 7 carbon atoms which, as will be appreciated, is attached to a terminal C═O group with a single bond (and may also be referred to as an “acyl group”). Alkanoyl groups include, but are not limited to, formyl, acetyl, propionyl, butyryl, isobutyryl, pentanoyl, isopentanoyl, 2-methyl-butyryl, 2,2-dimethoxypropionyl, hexanoyl, heptanoyl, octanoyl, and the like. “Lower alkanoyl” refers to an optionally substituted straight or branched aliphatic acyclic residue having about 1 to about 5 carbon atoms (and all combinations and subcombinations of ranges and specific number of carbon atoms). Such groups include, but are not limited to, formyl, acetyl, propionyl, butyryl, isobutyryl, pentanoyl, isopentanoyl, etc.

As used herein, “aryl” refers to an optionally substituted, mono- or bicyclic aromatic ring system having from about 5 to about 14 carbon atoms (and all combinations and subcombinations of ranges and specific numbers of carbon atoms therein), with from about 6 to about 10 carbons being preferred. Non-limiting examples include, for example, phenyl and naphthyl.

As used herein, “aralkyl” refers to alkyl radicals bearing an aryl substituent and have from about 6 to about 22 carbon atoms (and all combinations and subcombinations of ranges and specific numbers of carbon atoms therein), with from about 6 to about 12 carbon atoms being preferred in certain embodiments. Aralkyl groups can be optionally substituted. Non-limiting examples include, for example, benzyl, naphthylmethyl, diphenylmethyl, triphenylmethyl, phenylethyl, and diphenylethyl.

As used herein, the terms “alkoxy” and “alkoxyl” refer to an optionally substituted alkyl-O— group wherein alkyl is as previously defined. Exemplary alkoxy and alkoxyl groups include methoxy, ethoxy, n-propoxy, i-propoxy, n-butoxy, and heptoxy.

As used herein, “carboxy” refers to a —C(═O)OH group.

As used herein, “alkoxycarbonyl” refers to a —C(═O)O-alkyl group, where alkyl is as previously defined.

As used herein, “aroyl” refers to a —C(═O)-aryl group, wherein aryl is as previously defined. Exemplary aroyl groups include benzoyl and naphthoyl.

Typically, substituted chemical moieties include one or more substituents that replace hydrogen. Exemplary substituents include, for example, halo, alkyl, cycloalkyl, aralkyl, aryl, sulfhydryl, hydroxyl (—OH), alkoxyl, cyano (—CN), carboxyl (—COOH), —C(═O)O-alkyl, aminocarbonyl (—C(═O)NH₂), —N-substituted aminocarbonyl (—C(═O)NHR″), CF₃, CF₂CF₃, and the like. In relation to the aforementioned substituents, each moiety R″ can be, independently, any of H, alkyl, cycloalkyl, aryl, or aralkyl, for example.

As used herein, “L-amino acid” refers to any of the naturally occurring levorotatory alpha-amino acids normally present in proteins or the alkyl esters of those alpha-amino acids. The term D-amino acid” refers to dextrorotatory alpha-amino acids. Unless specified otherwise, all amino acids referred to herein are L-amino acids.

As used herein, an “aromatic amino acid” is an amino acid that comprises at least one aromatic ring, e.g., it comprises an aryl group.

As used herein, an “aromatic amino acid analog” is an amino acid analog that comprises at least one aromatic ring, e.g., it comprises an aryl group.

### DETAILED DESCRIPTION OF CERTAIN EMBODIMENTS OF THE INVENTION

The present invention provides methods of treating a subject in need of treatment for chronic rhinosinusitis, the methods comprising administering a complement inhibitor to the subject. In some embodiments of the invention, the complement inhibitor is a compstatin analog. The invention also provides methods of treating a subject in need of treatment for nasal polyposis, the methods comprising administering a complement inhibitor to the subject. In some embodiments of the invention, the complement inhibitor is a compstatin analog. As described in further detail below, compstatin analogs are complement inhibitors that bind to complement component C3 and inhibit its cleavage, thus inhibiting complement activation via the three major complement activation pathways. Compstatin analogs are highly effective in reducing formation of complement system effectors. The invention encompasses the recognition of the benefit of compstatin analogs in treating subjects suffering from chronic rhinosinusitis and/or nasal polyposis.

Chronic rhinosinusitis is a condition characterized by symptomatic inflammation of the paranasal sinuses (e.g., the maxillary, ethmoid, frontal, and/or sphenoidal sinuses) and nasal cavity. Symptoms and signs of CRS, and methods of diagnosis, are well known in the art. A diagnosis of CRS typically requires that signs and symptoms consistent with CRS have been present for at least 12 weeks. Diagnosis is usually based on symptoms and physical examination, e.g., rhinoscopic examination such as by anterior rhinoscopy and/or nasal endoscopy. Typical symptoms include nasal discharge, nasal obstruction (also termed blockage or congestion), facial pain and/or pressure, and/or decreased sense of smell. Typically, at least two symptoms are present.

Clinical diagnostic criteria for CRS have been developed by several organizations of medical/surgical practitioners and may be used as guidance. For example, the following criteria have been suggested by the American Academy of Otolaryngology Foundation: twelve (12) weeks or longer of two or more of the following signs and symptoms: (i) mucopurulent drainage (anterior, posterior, or both); (ii) nasal obstruction (congestion); (iii) facial pain-pressure-fullness; (iv) decreased sense of smell AND inflammation is documented by one or more of the following findings: (i) purulent (not clear) mucus or edema in the middle meatus or ethmoid region; (ii) polyps in nasal cavity or the middle meatus, and/or (iii) radiographic imaging showing inflammation of the paranasal sinuses. A position paper produced under the auspices of the European Academy of Allerology and Clinical Immunology suggests that two or more of the following symptoms should be present for at least 12 weeks for a diagnosis of CRS: (i) nasal blockage/obstruction/congestion; (ii) nasal discharge (anterior/posterior nasal drip); (iii) facial pain/pressure; (iv) reduction or loss of sense of smell, of which at least one of the symptoms must be nasal blockage/obstruction/congestion or nasal discharge. It will be appreciated that other diagnostic criteria can be used, and the diagnosis of CRS is within the discretion of the skilled practitioner.

Individuals with CRS may have acute exacerbations of CRS in which they experience worsening of the chronic baseline signs and symptoms or the development of new ones. These individuals do not have complete resolution of symptoms between exacerbations, thus distinguishing them from individuals with recurrent episodes of acute sinusitis (symptom duration <4 weeks) or sub-acute (symptom duration 4<12 weeks) sinusitis. In some embodiments of the present invention, a compstatin analog is administered to a subject suffering from or at risk of an exacerbation of CRS. In some embodiments, the compstatin analog is administered, e.g., until symptoms and/or signs of the exacerbation have substantially diminished or for a predetermined time period, e.g., 1-4 weeks. In some embodiments, the compstatin analog is administered prophylactically, e.g., if the subject has been exposed to a situation that may trigger an exacerbation of CRS, or if the subject is or may be experiencing the onset of an exacerbation.

While CRS is of particular interest herein, the invention also encompasses administration of compstatin analogs subjects suffering from acute or sub-acute rhinosinusitis, e.g., in combination with antiinfective therapy if appropriate. In some embodiments, a compstatin analog is administered to a subject with sub-acute rhinosinusitis. In some embodiments, the subject does not have bacterial sinusitis. In other embodiments, the subject has bacterial sinusitis.

A significant proportion of patients with CRS also have nasal polyposis, although nasal polyps can also arise in individuals who do not have CRS. Nasal polyps are nonmalignant lesions arising from the mucosa of the nasal sinuses (commonly at the outflow tract of one or more of the sinuses) or from the mucosa of the nasal cavity. They often originate from the mucosa of the middle meatus and clefts of the ethmoid region and can prolapse into the nasal cavity. Nasal polyps consist of loose connective tissue, edema, inflammatory cells, and some capillaries and glands. They can be covered with different types of epithelium, most commonly pseudostratified respiratory epithelium with goblet cells and ciliated cells. Polyp tissue is generally characterized by chronic eosinophilic infiltration. Other immune system cells such as lymphocytes, plasma cells, and mast cells are often present as well. See Assanasen, P and Robert M. Naclerio, M., Medical and surgical management of nasal polyps. Curr Opin Otolaryngol Head Neck Surg, 9:27-36, 2001, and references therein.

The main presenting symptom of NP is usually nasal obstruction which is often constant but can vary depending on the site and size of the polyps. Sufferers will also frequently complain of watery rhinorrhea and postnasal drip. Loss or reduced sense of smell, sometimes with a resulting alteration in taste, are also characteristic symptoms. Nasal and osteomeatal obstruction caused by nasal polyps may contribute to or help perpetuate purulent nasal discharge and CRS. Patients with CRS with nasal polyps appear on average to have more severe symptoms with less improvement after operative intervention, higher CT scores at presentation, and a significantly higher need for revision surgery as compared to CRS patients without nasal polyps (Deal R T & Kountakis S E. Significance of nasal polyps in chronic rhinosinusitis: symptoms and surgical outcomes. Laryngoscope. 114(11):1932-5, 2004).

CRS can occur together with a variety of other conditions. Immotile cilia disorders, and ciliary dyskinesia (e.g., due to Kartagener's syndrome) have been associated with an increased risk of CRS. Immune deficiency and anatomic abnormalities affecting the sinuses and/or nasal cavity (e.g., deviated nasal septum) can also predispose to CRS. In some embodiments of the invention, a compstatin analog is administered to a subject with CRS who does not have, e.g., has not been diagnosed, with a cilia disorder, immune deficiency, or anatomic abnormality.

Nasal polyps and CRS are often present in individuals with cystic fibrosis. In some embodiments, a compstatin analog is administered to a subject who has CF. In other embodiments, the subject does not have CF.

About 10% of persons with asthma have aspirin-induced asthma characterized by the triad of aspirin (or, more generally, NSAID) sensitivity, asthma, and nasal polyposis. NSAID sensitivity could be diagnosed based on history (e.g., association of symptoms with recent NSAID exposure) and/or antigen challenge. In certain embodiments of interest, a compstatin analog is administered to a subject who has been diagnosed with this triad. In other embodiments, a subject has not been diagnosed with the triad.

Allergic fungal sinusitis is sometimes associated with nasal polyposis. This condition may be diagnosed by the presence of a positive RAS test to fungus, NP, CT findings of hyperdense material in the sinus cavity, allergic mucus with histological evidence of eosinophilic preponderance, and/or identification of fungus in an appropriate sample (e.g., sinus mucus). In some embodiments of the invention, a compstatin analog is administered to a subject who has been diagnosed with allergic fungal sinusitis. In other embodiments, the subject has not been diagnosed with allergic fungal sinusitis.

Anterior rhinoscopy and nasal endoscopy are widely used in evaluating patients who may have CRS and/or NP and in monitoring response to therapy. Anterior rhinoscopy allows visualization of the anterior one-third of the nasal cavity with direct illumination and a speculum or other instrument to dilate the nasal vestibule. Nasal endoscopy can be performed with a flexible or rigid endoscope, typically after a topical decongestant and anesthetic are applied to the nasal mucosa. It involves placement of an endoscope inside the nose to capture images of the nasal cavity and sinus openings that are otherwise not visible by simple inspection of the nasal cavity with a nasal speculum and illumination. Nasal endoscopy also allows visualization of the posterior nasal cavity, nasopharynx, and sometimes the sinus drainage pathways in the middle meatus and superior meatus. Structures/spaces that may be visualized include the inferior turbinate, inferior meatus, and nasopharynx; sphenoethmoidal recess and sphenoidal ostium behind the middle and supreme turbinate; and middle meatus, including the uncinate process, hiatus semilunaris, maxillary ostia, nasofrontal recess, and anterior ethmoidal bulla. Nasal endoscopy allows identification of polyps or secretions in the posterior nasal cavity, within the middle meatus, or in the sphenoethmoidal recess. In addition, nasal endoscopy allows directed aspiration of abnormal secretions for analysis and culture, if desired.

Imaging studies such as plain X-rays, computed tomography (CT), and/or magnetic resonance imaging (MRI) may be of use in diagnosis, in evaluating severity or extent of disease, investigating treatment failures, etc. CT scan in subjects with CRS may reveal mucosal thickening, sinus opacification, and/or air/fluid level. CT scan allows evaluation of the extent of disease in subjects with NP and would almost always be performed if surgical treatment is considered

The severity of CRS and nasal polyposis can vary. A simple instrument to assess disease severity is to ask subjects to assign a score from 0 to 10 using a 10-point scoring system or visual analog scale. In one embodiment, a visual analogue scale is a continuous horizontal line of 10 cm where: 0 cm indicates no complaints and 10 cm indicates severe complaints (see, e.g., Blom H M, et al., Clin Exp Allergy 1997; 27:796-801). The subject is asked to evaluate complaints over a preceding time period, e.g., the past 14 days. The disease severity can be classified as mild (0-3), moderate (4-7), and severe (8-10).

A variety of objective measures suitable for assessing, e.g., quantifying, the severity of CRS and/or nasal polyposis are available. For example, olfactory function can be assessed using the Smell Identification Test (Doty R L. The Smell Identification Test™ Administration Manual. 3rd ed. Sensonics, Inc.; Haddon Heights, N.J., pp. 1-17, 1995). The Lund-Mackay CT score (Lund V J, Mackay I S. Staging in rhinosinusitis. Rhinology. 107:183-184, 1993) and/or Lund-Kennedy endoscopy score (Lund V J, Kennedy D W. Quantification for staging sinusitis. International Conference on Sinus Disease: terminology, staging, therapy. Ann Oto Rhinol Laryngol. 104(Suppl):17-21, 1995). The Lund-Mackay system assigns a score of 0-2 dependent on the absence, partial, or complete opacification of each sinus system and of the ostiomeatal complex, deriving a maximum score of 12 per side.

Nasal polyps can be graded based on size and/or number. A variety of grading methods, e.g., based on endoscopy and/or CT, are available for assessment of nasal polyposis. For example, these include: (1) lateral imaging projecting the extension of the polyps by drawing on a schematic picture of the lateral wall of each nasal cavity; (2) assessment of polyp obstruction estimating the proportion of the total nasal cavity volume occupied by polyps; (3) nasal airway patency—determining the relationship between the patient's patent airway lumen and an imaginary maximal nasal airway lumen; (4) a four point scoring system of Lildholdt et al., involving determining their relationship to fixed anatomical landmarks; and (5) a three point scoring system of Lund and Mackay (see, e.g., Johansson L, Acta Otolaryngol., 120(1):72-6, 2000, and references therein). For example, polyps can be scored as 1, 2, or 3 (on a scale of 0 to 3) in each of the right and left nasal cavities, where 0 indicates no polyps; 1, polyps within the middle meatus; 2, polyps not confined to the middle meatus; and 3, completely obstructive polyps; or where 0 indicates no polyps, 1, polyps restricted to middle meatus; 2, polyps below middle turbinate; and 3, massive polyposis.

Validated, disease-specific quality of life (QOL) instruments, such as the Rhinosinusitis Disability Index (RSDI), the Chronic Sinusitis Survey (CSS), or the Sinonasal Outcomes Test (SNOT) can be used. The RSDI is a 30-question survey (range: 0-120) developed to predict rhinosinusitis specific health outcomes in three domains (physical, functional, and emotional) (Benninger M S, Senior B A. The development of the rhinosinusitis disability index. Arch Otolaryngol Head Neck Surg. 123:1175-1179, 1997). Higher scores on the RSDI represent a higher level of disease impact and worse QOL status. The CSS is a 6 item disease-specific questionnaire developed for assessing health status and treatment effectiveness in CRS. Higher total and subscale scores (range: 0-100) represent a lower impact of disease and better QOL status (Gliklich R E, Metson R. Techniques for outcomes research in chronic rhinosinusitis. Laryngoscope. 105:387-390, 1995). The 20-Item Sino-Nasal Outcome Test (SNOT-20) is a validated, self-administered, quality of life instrument specific for patients with symptoms of rhinosinusitis (Piccirillo J F, et al. Psychometric and clinimetric validity of the 20-Item Sino-Nasal Outcome Test (SNOT-20). Otolaryngol Head Neck Surg. 126(1):41-47, 2002). The instrument measures physical problems, functional limitations, and emotional consequences of sinusitis by asking subjects to score 20 items, including the need to blow the nose, sneezing, runny nose, cough, postnasal discharge, thick nasal discharge, ear fullness, dizziness, ear pain, facial pain/pressure, difficulty falling asleep, waking up at night, lack of a good night's sleep, waking up tired, fatigue, reduced productivity, reduced concentration, frustrated/restless/irritable, and being sad and embarrassed. The more recently developed 22 item Sinonasal Outcome Test (SNOT-22), a modification of the SNOT-20, can be used (see, e.g., Hopkins C., et al. Psychometric validity of the 22-item Sinonasal Outcome Test. Clin Otolaryngol., 34(5):447-54, 2009). The Medical Short Form-36 is a general health-related QOL instrument. The SF-36 contains measures overall QOL status in eight individual domains: general health, physical functioning, physical role, bodily pain, vitality, social functioning, emotional role, and mental health. Higher subscale scores (range: 0-100) represent a lower impact of disease severity and better overall health.

Complement System

To facilitate understanding the invention, a brief, non-limiting description of the complement system is provided. Complement is an arm of the innate immune system that plays an important role in defending the body against infectious agents. The complement system comprises more than 30 serum and cellular proteins that are involved in three major pathways: the classical, alternative, and lectin pathways. The classical pathway is usually triggered by binding of a complex of antigen and IgM or IgG antibody to C1. Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase. In the alternative pathway, C3b, resulting from cleavage of C3, which occurs spontaneously at a low level, binds to certain targets such as microbial cell surfaces or various complex polysaccharides and forms a complex with factor B, which is later cleaved by factor D, resulting in a C3 convertase. Cleavage of C3 and binding of another molecule of C3b to the C3 convertase gives rise to a C5 convertase. The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase and subsequent reactions as described above.

Complement effectors include the MAC, C3a, C4a, and C5a. The MAC inserts itself into target cell membranes and causes cell lysis. Small amounts of MAC on the membrane of cells may have a variety of consequences other than cell death. C3a, C4a, and C5a are anaphylotoxins and mediate multiple reactions in the acute inflammatory response including acting as chemotactic factors for immune system cells such as neutrophils.

Complement activity is normally regulated by a number of endogenous soluble or cell surface proteins. The complement control protein (CCP) family includes complement receptor type 1 (CR1; C3b:C4b receptor), complement receptor type 2 (CR2), membrane cofactor protein (MCP; CD46), decay-accelerating factor (DAF), complement factor H (1H), and C4b-binding protein (C4bp). CD59 is a membrane-bound complement regulatory protein unrelated structurally to the CCPs. Further details regarding complement are found in, e.g., Kuby Immunology, 6^{th} ed., 2006; Paul, W. E., Fundamental Immunology, Lippincott Williams & Wilkins; 6^{th} ed., 2008; and Walport M J., Complement. First of two parts. N Engl J Med., 344(14):1058-66, 2001.

Compstatin Analogs

Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 1), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7(3):619-27, 1998) to refer to a peptide having the same sequence as SEQ ID NO: 2 disclosed in U.S. Pat. No. 6,319,897, but amidated at the C terminus as shown in Table 1 (SEQ ID NO: 8). The term “compstatin” is used herein consistently with such usage (i.e., to refer to SEQ ID NO: 8). Compstatin analogs that have higher complement inhibiting activity than compstatin have been developed. See, e.g., WO2004/026328 (PCT/US2003/029653), Morikis, D., et al., Biochem Soc Trans. 32(Pt 1):28-32, 2004, Mallik, B., et al., J. Med. Chem., 274-286, 2005; Katragadda, M., et al. J. Med. Chem., 49: 4616-4622, 2006; WO2007062249 (PCT/US2006/045539); WO2007044668 (PCT/US2006/039397), PCT/US2008/078593 (WO/2009/046198), WO/2010/127336 (PCT/US2010/033345) and discussion below.

Compstatin analogs may be acetylated or amidated, e.g., at the N-terminus and/or C-terminus. For example, compstatin analogs may be acetylated at the N-terminus and amidated at the C-terminus. Consistent with usage in the art, “compstatin” as used herein, and the activities of compstatin analogs described herein relative to that of compstatin, refer to compstatin amidated at the C-terminus (Mallik, 2005, supra).

Concatamers or multimers of compstatin or a complement inhibiting analog thereof are also of use in the present invention.

As used herein, the term “compstatin analog” includes compstatin and any complement inhibiting analog thereof. The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. Certain compstatin analogs and suitable assays are described in U.S. Pat. No. 6,319,897, WO2004/026328, Morikis, supra, Mallik, supra, Katragadda 2006, supra, WO2007062249 (PCT/US2006/045539); WO2007044668 (PCT/US2006/039397), WO/2009/046198 (PCT/US2008/078593); and/or WO/2010/127336 (PCT/US2010/033345). The assay may, for example, measure alternative or classical pathway-mediated erythrocyte lysis or be an ELISA assay (see, e.g., Examples 4 and 5 of U.S. Ser. No. 11/544,389). In some embodiments, an assay described in WO/2010/135717 (PCT/US2010/035871) is used. The invention includes embodiments in which any one or more of the compstatin analogs or compositions described herein is used in any the methods of treatment described herein.

The activity of a compstatin analog may be expressed in terms of its IC₅₀ (the concentration of the compound that inhibits complement activation by 50%), with a lower IC₅₀ indicating a higher activity as recognized in the art. The activity of a preferred compstatin analog for use in the present invention is at least as great as that of compstatin. It is noted that certain modifications are known to reduce or eliminate complement inhibiting activity and may be explicitly excluded from any embodiment of the invention. The IC₅₀ of compstatin has been measured as 12 μM using an alternative pathway-mediated erythrocyte lysis assay (WO2004/026328). It will be appreciated that the precise IC₅₀ value measured for a given compstatin analog will vary with experimental conditions (e.g., the serum concentration used in the assay). Comparative values, e.g., obtained from experiments in which IC₅₀ is determined for multiple different compounds under substantially identical conditions, are of use. In one embodiment, the IC₅₀ of the compstatin analog is no more than the IC₅₀ of compstatin. In certain embodiments of the invention the activity of the compstatin analog is between 2 and 99 times that of compstatin (i.e., the analog has an IC₅₀ that is less than the IC₅₀ of compstatin by a factor of between 2 and 99). For example, the activity may be between 10 and 50 times as great as that of compstatin, or between 50 and 99 times as great as that of compstatin. In certain embodiments of the invention the activity of the compstatin analog is between 99 and 264 times that of compstatin. For example, the activity may be 100, 110, 120, 130, 140, 150, 160, 170, 180, 190, 200, 210, 220, 230, 240, 250, 260, or 264 times as great as that of compstatin. In certain embodiments the activity is between 250 and 300, 300 and 350, 350 and 400, or 400 and 500 times as great as that of compstatin. The invention further contemplates compstatin analogs having activities between 500 and 1000 times that of compstatin, or more, e.g., between 1000 and 2000 times that of compstatin, or more. In certain embodiments the IC₅₀ of the compstatin analog is between about 0.2 μM and about 0.5 μM. In certain embodiments the IC₅₀ of the compstatin analog is between about 0.1 μM and about 0.2 μM. In certain embodiments the IC₅₀ of the compstatin analog is between about 0.05 μM and about 0.1 μM. In certain embodiments the IC₅₀ of the compstatin analog is between about 0.001 μM and about 0.05 μM.

The K_{d} of compstatin binding to C3 can be measured using isothermal titration calorimetry (Katragadda, et al., J. Biol. Chem., 279(53), 54987-54995, 2004). Binding affinity of a variety of compstatin analogs for C3 has been correlated with their activity, with a lower K_{d} indicating a higher binding affinity, as recognized in the art. A linear correlation between binding affinity and activity was shown for certain analogs tested (Katragadda, 2004, supra; Katragadda 2006, supra). In certain embodiments of the invention the compstatin analog binds to C3 with a K_{d} of between 0.1 μM and 1.0 μM, between 0.05 μM and 0.1 μM, between 0.025 μM and 0.05 μM, between 0.015 μM and 0.025 μM, between 0.01 μM and 0.015 μM, or between 0.001 μM and 0.01 μM.

Compounds “designed or identified based on compstatin” include, but are not limited to, compounds that comprise an amino acid chain whose sequence is obtained by (i) modifying the sequence of compstatin (e.g., replacing one or more amino acids of the sequence of compstatin with a different amino acid or amino acid analog, inserting one or more amino acids or amino acid analogs into the sequence of compstatin, or deleting one or more amino acids from the sequence of compstatin); (ii) selection from a phage display peptide library in which one or more amino acids of compstatin is randomized, and optionally further modified according to method (i); or (iii) identified by screening for compounds that compete with compstatin or any analog thereof obtained by methods (i) or (ii) for binding to C3 or a fragment thereof. Many useful compstatin analogs comprise a hydrophobic cluster, a β-turn, and a disulfide bridge.

In certain embodiments of the invention the sequence of the compstatin analog comprises or consists essentially of a sequence that is obtained by making 1, 2, 3, or 4 substitutions in the sequence of compstatin, i.e., 1, 2, 3, or 4 amino acids in the sequence of compstatin is replaced by a different standard amino acid or by a non-standard amino acid. In certain embodiments of the invention the amino acid at position 4 is altered. In certain embodiments of the invention the amino acid at position 9 is altered. In certain embodiments of the invention the amino acids at positions 4 and 9 are altered. In certain embodiments of the invention only the amino acids at positions 4 and 9 are altered. In certain embodiments of the invention the amino acid at position 4 or 9 is altered, or in certain embodiments both amino acids 4 and 9 are altered, and in addition up to 2 amino acids located at positions selected from 1, 7, 10, 11, and 13 are altered. In certain embodiments of the invention the amino acids at positions 4, 7, and 9 are altered. In certain embodiments of the invention amino acids at position 2, 12, or both are altered, provided that the alteration preserves the ability of the compound to be cyclized. Such alteration(s) at positions 2 and/or 12 may be in addition to the alteration(s) at position 1, 4, 7, 9, 10, 11, and/or 13. Optionally the sequence of any of the compstatin analogs whose sequence is obtained by replacing one or more amino acids of compstatin sequence further includes up to 1, 2, or 3 additional amino acids at the C-terminus. In one embodiment, the additional amino acid is Gly. Optionally the sequence of any of the compstatin analogs whose sequence is obtained by replacing one or more amino acids of compstatin sequence further includes up to 5, or up to 10 additional amino acids at the C-terminus. It should be understood that compstatin analogs may have any one or more of the characteristics or features of the various embodiments described herein, and characteristics or features of any embodiment may additionally characterize any other embodiment described herein, unless otherwise stated or evident from the context. In certain embodiments of the invention the sequence of the compstatin analog comprises or consists essentially of a sequence identical to that of compstatin except at positions corresponding to positions 4 and 9 in the sequence of compstatin.

Compstatin and certain compstatin analogs having somewhat greater activity than compstatin contain only standard amino acids (“standard amino acids” are glycine, leucine, isoleucine, valine, alanine, phenylalanine, tyrosine, tryptophan, aspartic acid, asparagine, glutamic acid, glutamine, cysteine, methionine, arginine, lysine, proline, serine, threonine and histidine). Certain compstatin analogs having improved activity incorporate one or more non-standard amino acids. Useful non-standard amino acids include singly and multiply halogenated (e.g., fluorinated) amino acids, D-amino acids, homo-amino acids, N-alkyl amino acids, dehydroamino acids, aromatic amino acids (other than phenylalanine, tyrosine and tryptophan), ortho-, meta- or para-aminobenzoic acid, phospho-amino acids, methoxylated amino acids, and α,α-disubstituted amino acids. In certain embodiments of the invention, a compstatin analog is designed by replacing one or more L-amino acids in a compstatin analog described elsewhere herein with the corresponding D-amino acid. Such compounds and methods of use thereof are an aspect of the invention. Exemplary non-standard amino acids of use include 2-naphthylalanine (2-NaI), 1-naphthylalanine (1-NaI), 2-indanylglycine carboxylic acid (2Ig1), dihydrotrpytophan (Dht), 4-benzoyl-L-phenylalanine (Bpa), 2-α-aminobutyric acid (2-Abu), 3-α-aminobutyric acid (3-Abu), 4-α-aminobutyric acid (4-Abu), cyclohexylalanine (Cha), homocyclohexylalanine (hCha), 4-fluoro-L-tryptophan (4fW), 5-fluoro-L-tryptophan (5fW), 6-fluoro-L-tryptophan (6fW), 4-hydroxy-L-tryptophan (4OH—W), 5-hydroxy-L-tryptophan (5OH—W), 6-hydroxy-L-tryptophan (6OH—W), 1-methyl-L-tryptophan (1MeW), 4-methyl-L-tryptophan (4MeW), 5-methyl-L-tryptophan (5MeW), 7-aza-L-tryptophan (7aW), α-methyl-L-tryptophan (αMeW), β-methyl-L-tryptophan (βMeW), N-methyl-L-tryptophan (NMeW), ornithine (orn), citrulline, norleucine, γ-glutamic acid, etc.

In certain embodiments of the invention the compstatin analog comprises one or more Trp analogs (e.g., at position 4 and/or 7 relative to the sequence of compstatin). Exemplary Trp analogs are mentioned above. See also Beene, et. al. Biochemistry 41: 10262-10269, 2002 (describing, inter alia, singly- and multiply-halogenated Trp analogs); Babitzke & Yanofsky, J. Biol. Chem. 270: 12452-12456, 1995 (describing, inter alia, methylated and halogenated Trp and other Trp and indole analogs); and U.S. Pat. Nos. 6,214,790, 6,169,057, 5,776,970, 4,870,097, 4,576,750 and 4,299,838. Other Trp analogs include variants that are substituted (e.g., by a methyl group) at the α or β carbon and, optionally, also at one or more positions of the indole ring. Amino acids comprising two or more aromatic rings, including substituted, unsubstituted, or alternatively substituted variants thereof, are of interest as Trp analogs. In certain embodiments of the invention the Trp analog, e.g., at position 4, is 5-methoxy, 5-methyl-, 1-methyl-, or 1-formyl-tryptophan. In certain embodiments of the invention a Trp analog (e.g., at position 4) comprising a 1-alkyl substituent, e.g., a lower alkyl (e.g., C₁-C₅) substituent is used. In certain embodiments, N(α) methyl tryptophan or 5-methyltryptophan is used. In some embodiments, an analog comprising a 1-alkanyol substituent, e.g., a lower alkanoyl (e.g., C₁-C₅) is used. In some embodiments, the analog is 1-acetyl-L-tryptophan or L-β-homo-tryptophan.

In certain embodiments the Trp analog has increased hydrophobic character relative to Trp. For example, the indole ring may be substituted by one or more alkyl (e.g., methyl) groups. In certain embodiments the Trp analog participates in a hydrophobic interaction with C3. Such a Trp analog may be located, e.g., at position 4 relative to the sequence of compstatin. In certain embodiments the Trp analog comprises a substituted or unsubstituted bicyclic aromatic ring component or two or more substituted or unsubstituted monocyclic aromatic ring components.

In certain embodiments the Trp analog has increased propensity to form hydrogen bonds with C3 relative to Trp but does not have increased hydrophobic character relative to Trp. The Trp analog may have increased polarity relative to Trp and/or an increased ability to participate in an electrostatic interaction with a hydrogen bond donor on C3. Certain exemplary Trp analogs with an increased hydrogen bond forming character comprise an electronegative substituent on the indole ring. Such a Trp analog may be located, e.g., at position 7 relative to the sequence of compstatin.

In certain embodiments of the invention the compstatin analog comprises one or more Ala analogs (e.g., at position 9 relative to the sequence of compstatin), e.g., Ala analogs that are identical to Ala except that they include one or more CH₂ groups in the side chain. In certain embodiments the Ala analog is an unbranched single methyl amino acid such as 2-Abu. In certain embodiments of the invention the compstatin analog comprises one or more Trp analogs (e.g., at position 4 and/or 7 relative to the sequence of compstatin) and an Ala analog (e.g., at position 9 relative to the sequence of compstatin).

In certain embodiments of the invention the compstatin analog is a compound that comprises a peptide that has a sequence of (X′aa)ₙ-Gln-Asp-Xaa-Gly-(X″aa)ₘ, (SEQ ID NO: 2) wherein each X′aa and each X″aa is an independently selected amino acid or amino acid analog, wherein Xaa is Trp or an analog of Trp, and wherein n>1 and m>1 and n+m is between 5 and 21. The peptide has a core sequence of Gln-Asp-Xaa-Gly, where Xaa is Trp or an analog of Trp, e.g., an analog of Trp having increased propensity to form hydrogen bonds with an H-bond donor relative to Trp but, in certain embodiments, not having increased hydrophobic character relative to Trp. For example, the analog may be one in which the indole ring of Trp is substituted with an electronegative moiety, e.g., a halogen such as fluorine. In one embodiment Xaa is 5-fluorotryptophan. Absent evidence to the contrary, one of skill in the art would recognize that any non-naturally occurring peptide whose sequence comprises this core sequence and that inhibits complement activation and/or binds to C3 will have been designed based on the sequence of compstatin. In an alternative embodiment Xaa is an amino acid or amino acid analog other than a Trp analog that allows the Gln-Asp-Xaa-Gly peptide to form a β-turn.

In certain embodiments of the invention the peptide has a core sequence of X′aa-Gln-Asp-Xaa-Gly (SEQ ID NO: 3), where X′aa and Xaa are selected from Trp and analogs of Trp. In certain embodiments of the invention the peptide has a core sequence of X′aa-Gln-Asp-Xaa-Gly (SEQ ID NO: 3), where X′aa and Xaa are selected from Trp, analogs of Trp, and other amino acids or amino acid analogs comprising at least one aromatic ring. In certain embodiments of the invention the core sequence forms a β-turn in the context of the peptide. The β-turn may be flexible, allowing the peptide to assume two or more conformations as assessed for example, using nuclear magnetic resonance (NMR). In certain embodiments X′aa is an analog of Trp that comprises a substituted or unsubstituted bicyclic aromatic ring component or two or more substituted or unsubstituted monocyclic aromatic ring components. In certain embodiments of the invention X′aa is selected from the group consisting of 2-napthylalanine, 1-napthylalanine, 2-indanylglycine carboxylic acid, dihydrotryptophan, and benzoylphenylalanine. In certain embodiments of the invention X′aa is an analog of Trp that has increased hydrophobic character relative to Trp. For example, X′aa may be 1-methyltryptophan. In certain embodiments of the invention Xaa is an analog of Trp that has increased propensity to form hydrogen bonds relative to Trp but, in certain embodiments, not having increased hydrophobic character relative to Trp. In certain embodiments of the invention the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan.

In certain embodiments of the invention the peptide has a core sequence of X′aa-Gln-Asp-Xaa-Gly-X″aa (SEQ ID NO: 4), where X′aa and Xaa are each independently selected from Trp and analogs of Trp and X″aa is selected from His, Ala, analogs of Ala, Phe, and Trp. In certain embodiments of the invention X′aa is an analog of Trp that has increased hydrophobic character relative to Trp, such as 1-methyltryptophan or another Trp analog having an alkyl substituent on the indole ring (e.g., at position 1, 4, 5, or 6). In certain embodiments X′aa is an analog of Trp that comprises a substituted or unsubstituted bicyclic aromatic ring component or two or more substituted or unsubstituted monocyclic aromatic ring components. In certain embodiments of the invention X′aa is selected from the group consisting of 2-napthylalanine, 1-napthylalanine, 2-indanylglycine carboxylic acid, dihydrotryptophan, and benzoylphenylalanine. In certain embodiments of the invention Xaa is an analog of Trp that has increased propensity to form hydrogen bonds with C3 relative to Trp but, in certain embodiments, not having increased hydrophobic character relative to Trp. In certain embodiments of the invention the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan. In certain embodiments X″aa is Ala or an analog of Ala such as Abu or another unbranched single methyl amino acid. In certain embodiments of the invention the peptide has a core sequence of X′aa-Gln-Asp-Xaa-Gly-X″aa (SEQ ID NO: 4), where X′aa and Xaa are each independently selected from Trp, analogs of Trp, and amino acids or amino acid analogs comprising at least one aromatic side chain, and X″aa is selected from His, Ala, analogs of Ala, Phe, and Trp. In certain embodiments X″aa is selected from analogs of Trp, aromatic amino acids, and aromatic amino acid analogs.

In certain preferred embodiments of the invention the peptide is cyclic. The peptide may be cyclized via a bond between any two amino acids, one of which is (X′aa)ₙ and the other of which is located within (X″aa)ₘ. In certain embodiments the cyclic portion of the peptide is between 9 and 15 amino acids in length, e.g., 10-12 amino acids in length. In certain embodiments the cyclic portion of the peptide is 11 amino acids in length, with a bond (e.g., a disulfide bond) between amino acids at positions 2 and 12. For example, the peptide may be 13 amino acids long, with a bond between amino acids at positions 2 and 12 resulting in a cyclic portion 11 amino acids in length.

In certain embodiments the peptide comprises or consists of the sequence X′aa1-X′aa2-X′aa3-X′aa4-Gln-Asp-Xaa-Gly-X″aa1-X″aa2-X″aa3-X″aa4-X″aa5 (SEQ ID NO: 5). In certain embodiments X′aa4 and Xaa are selected from Trp and analogs of Trp, and X′aa1, X′aa2, X′aa3, X″aa1, X″aa2, X″aa3, X″aa4, and X″aa5 are independently selected from among amino acids and amino acid analogs. In certain embodiments X′aa4 and Xaa are selected from aromatic amino acids and aromatic amino acid analogs. Any one or more of X′aa1, X′aa2, X′aa3, X″aa1, X″aa2, X″aa3, X″aa4, and X″aa5 may be identical to the amino acid at the corresponding position in compstatin. In one embodiment, X″aa1 is Ala or a single methyl unbranched amino acid. The peptide may be cyclized via a covalent bond between (i) X′aa1, X′aa2, or X′aa3; and (ii) X″aa2, X″aa3, X″aa4 or X″aa5. In one embodiment the peptide is cyclized via a covalent bond between X′aa2 and X″aa4. In one embodiment the covalently bound amino acid are each Cys and the covalent bond is a disulfide (S—S) bond. In other embodiments the covalent bond is a C—C, C—O, C—S, or C—N bond. In certain embodiments one of the covalently bound residues is an amino acid or amino acid analog having a side chain that comprises a primary or secondary amine, the other covalently bound residue is an amino acid or amino acid analog having a side chain that comprises a carboxylic acid group, and the covalent bond is an amide bond. Amino acids or amino acid analogs having a side chain that comprises a primary or secondary amine include lysine and diaminocarboxylic acids of general structure NH₂(CH₂)ₙCH(NH₂)COOH such as 2,3-diaminopropionic acid (dapa), 2,4-diaminobutyric acid (daba), and ornithine (orn), wherein n=1 (dapa), 2 (daba), and 3 (orn), respectively. Examples of amino acids having a side chain that comprises a carboxylic acid group include dicarboxylic amino acids such as glutamic acid and aspartic acid. Analogs such as beta-hydroxy-L-glutamic acid may also be used.

In certain embodiments, the compstatin analog is a compound that comprises a peptide having a sequence:

Xaa1-Cys-Val-Xaa2-Gln-Asp-Xaa2*-Gly-Xaa3-His-Arg-Cys-Xaa4 (SEQ ID NO: 6); wherein:

Xaa1 is Ile, Val, Leu, B¹-Ile, B¹-Val, B¹-Leu or a dipeptide comprising Gly-Ile or B¹-Gly-Ile, and B¹ represents a first blocking moiety;
Xaa2 and Xaa2* are independently selected from Trp and analogs of Trp;
Xaa3 is His, Ala or an analog of Ala, Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of the L-Thr, D-Thr, Ile, Val, Gly, Ala, or Asn optionally is replaced by a second blocking moiety B²; and the two Cys residues are joined by a disulfide bond. In some embodiments, Xaa4 is Leu, Nle, His, or Phe or a dipeptide selected from Xaa5-Ala and Xaa5-Asn, or a tripeptide Xaa5-Ala-Asn, wherein Xaa5 is selected from Leu, Nle, His or Phe, and wherein a carboxy terminal —OH of any of the L-Thr, D-Thr, Ile, Val, Gly, Leu, Nle, His, Phe, Ala, or Asn optionally is replaced by a second blocking moiety B²; and the two Cys residues are joined by a disulfide bond.

In other embodiments Xaa1 is absent or is any amino acid or amino acid analog, and Xaa2, Xaa2*, Xaa3, and Xaa4 are as defined above. If Xaa1 is absent, the N-terminal Cys residue may have a blocking moiety B¹ attached thereto.

In another embodiment, Xaa4 is any amino acid or amino acid analog and Xaa1, Xaa2, Xaa2*, and Xaa3 are as defined above. In another embodiment Xaa4 is a dipeptide selected from the group consisting of: Thr-Ala and Thr-Asn, wherein the carboxy terminal —OH or the Ala or Asn is optionally replaced by a second blocking moiety B².

In any of the embodiments of the compstatin analog of SEQ ID NO: 6, Xaa2 may be Trp.

In any of the embodiments of the compstatin analog of SEQ ID NO: 6, Xaa2 may be an analog of Trp comprising a substituted or unsubstituted bicyclic aromatic ring component or two or more substituted or unsubstituted monocyclic aromatic ring components. For example, the analog of Trp may be selected from 2-naphthylalanine (2-NaI), 1-naphthylalanine (1-NaI), 2-indanylglycine carboxylic acid (Ig1), dihydrotrpytophan (Dht), and 4-benzoyl-L-phenylalanine.

In any of the embodiments of the compstatin analog of SEQ ID NO: 6, Xaa2 may be an analog of Trp having increased hydrophobic character relative to Trp. For example, the analog of Trp may be selected from 1-methyltryptophan, 4-methyltryptophan, 5-methyltryptophan, and 6-methyltryptophan. In one embodiment, the analog of Trp is 1-methyltryptophan. In one embodiment, Xaa2 is 1-methyltryptophan, Xaa2* is Trp, Xaa3 is Ala, and the other amino acids are identical to those of compstatin.

In any of the embodiments of the compstatin analog of SEQ ID NO: 6, Xaa2* may be an analog of Trp such as an analog of Trp having increased hydrogen bond forming propensity with C3 relative to Trp, which, in certain embodiments, does not have increased hydrophobic character relative to Trp. In certain embodiments the analog of Trp comprises an electronegative substituent on the indole ring. For example, the analog of Trp may be selected from 5-fluorotryptophan and 6-fluorotryptophan.

In certain embodiments of the invention Xaa2 is Trp and Xaa2* is an analog of Trp having increased hydrogen bond forming propensity with C3 relative to Trp which, in certain embodiments, does not have increased hydrophobic character relative to Trp. In certain embodiments of the compstatin analog of SEQ ID NO: 6, Xaa2 is analog of Trp having increased hydrophobic character relative to Trp such as an analog of Trp selected from 1-methyltryptophan, 4-methyltryptophan, 5-methyltryptophan, and 6-methyltryptophan, and Xaa2* is an analog of Trp having increased hydrogen bond forming propensity with C3 relative to Trp which, in certain embodiments, does not have increased hydrophobic character relative to Trp. For example, in one embodiment Xaa2 is methyltryptophan and Xaa2* is 5-fluorotryptophan.

In certain of the afore-mentioned embodiments, Xaa3 is Ala. In certain of the afore-mentioned embodiments Xaa3 is a single methyl unbranched amino acid, e.g., Abu.

The invention further provides compstatin analogs of SEQ ID NO: 6, as described above, wherein Xaa2 and Xaa2* are independently selected from Trp, analogs of Trp, and other amino acids or amino acid analogs that comprise at least one aromatic ring, and Xaa3 is His, Ala or an analog of Ala, Phe, Trp, an analog of Trp, or another aromatic amino acid or aromatic amino acid analog.

In certain embodiments of the invention the blocking moiety present at the N- or C-terminus of any of the compstatin analogs described herein is any moiety that stabilizes a peptide against degradation that would otherwise occur in mammalian (e.g., human or non-human primate) blood or interstitial fluid. For example, blocking moiety B¹ could be any moiety that alters the structure of the N-terminus of a peptide so as to inhibit cleavage of a peptide bond between the N-terminal amino acid of the peptide and the adjacent amino acid. Blocking moiety B² could be any moiety that alters the structure of the C-terminus of a peptide so as to inhibit cleavage of a peptide bond between the C-terminal amino acid of the peptide and the adjacent amino acid. Any suitable blocking moieties known in the art could be used. In certain embodiments of the invention blocking moiety B¹ comprises an acyl group (i.e., the portion of a carboxylic acid that remains following removal of the —OH group). The acyl group typically comprises between 1 and 12 carbons, e.g., between 1 and 6 carbons. For example, in certain embodiments of the invention blocking moiety B¹ is selected from the group consisting of: formyl, acetyl, proprionyl, butyryl, isobutyryl, valeryl, isovaleryl, etc. In one embodiment, the blocking moiety B¹ is an acetyl group, i.e., Xaa1 is Ac-Ile, Ac-Val, Ac-Leu, or Ac-Gly-Ile.

In certain embodiments of the invention blocking moiety B² is a primary or secondary amine (—NH₂ or —NHR¹, wherein R is an organic moiety such as an alkyl group).

In certain embodiments of the invention blocking moiety B¹ is any moiety that neutralizes or reduces the negative charge that may otherwise be present at the N-terminus at physiological pH. In certain embodiments of the invention blocking moiety B² is any moiety that neutralizes or reduces the negative charge that may otherwise be present at the C-terminus at physiological pH.

In certain embodiments of the invention, the compstatin analog is acetylated or amidated at the N-terminus and/or C-terminus, respectively. A compstatin analog may be acetylated at the N-terminus, amidated at the C-terminus, and or both acetylated at the N-terminus and amidated at the C-terminus. In certain embodiments of the invention a compstatin analog comprises an alkyl or aryl group at the N-terminus rather than an acetyl group.

In certain embodiments, the compstatin analog is a compound that comprises a peptide having a sequence:

Xaa1-Cys-Val-Xaa2-Gln-Asp-Xaa2*-Gly-Xaa3-His-Arg-Cys-Xaa4 (SEQ ID NO: 7); wherein:

Xaa1 is Ile, Val, Leu, Ac-Ile, Ac-Val, Ac-Leu or a dipeptide comprising Gly-Ile or Ac-Gly-Ile;
Xaa2 and Xaa2* are independently selected from Trp and analogs of Trp;
Xaa3 is His, Ala or an analog of Ala, Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of L-Thr, D-Thr, Ile, Val, Gly, Ala, or Asn optionally is replaced by —NH₂; and
the two Cys residues are joined by a disulfide bond. In some embodiments, Xaa4 is Leu, Nle, His, or Phe or a depeptide selected from Xaa5-Ala and Xaa5-Asn, or a tripeptide Xaa5-Ala-Asn, wherein Xaa5 is selected from Leu, Nle, His or Phe, and wherein a carboxy terminal —OH of any of the L-Thr, D-Thr, Ile, Val, Gly, Leu, Nle, His, Phe, Ala, or Asn optionally is replaced by a second blocking moiety B2; and the two Cys residues are joined by a disulfide bond.

In some embodiments, Xaa1, Xaa2, Xaa2*, Xaa3, and Xaa4 are as described above for the various embodiments of SEQ ID NO: 6. For example, in certain embodiments Xaa2* is Trp. In certain embodiments Xaa2 is an analog of Trp having increased hydrophobic character relative to Trp, e.g., 1-methyltryptophan. In certain embodiments Xaa3 is Ala. In certain embodiments Xaa3 is a single methyl unbranched amino acid.

In certain embodiments of the invention Xaa1 is Ile and Xaa4 is L-Thr.

In certain embodiments of the invention Xaa1 is Ile, Xaa2* is Trp, and Xaa4 is L-Thr.

The invention further provides compstatin analogs of SEQ ID NO: 7, as described above, wherein Xaa2 and Xaa2* are independently selected from Trp, analogs of Trp, other amino acids or aromatic amino acid analogs, and

Xaa3 is His, Ala or an analog of Ala, Phe, Trp, an analog of Trp, or another aromatic amino acid or aromatic amino acid analog.

In certain embodiments of any of the compstatin analogs described herein, an analog of Phe is used rather than Phe.

Table 1 provides a non-limiting list of compstatin analogs useful in the present invention. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus. Unless otherwise indicated, peptides in Table 1 are amidated at the C-terminus Bold text is used to indicate certain modifications. Activity relative to compstatin is based on published data and assays described therein (WO2004/026328, WO2007044668, Mallik, 2005; Katragadda, 2006). Where multiple publications reporting an activity were consulted, the more recently published value is used, and it will be recognized that values may be adjusted in the case of differences between assays. It will also be appreciated that in certain embodiments of the invention the peptides listed in Table 1 are cyclized via a disulfide bond between the two Cys residues when used in the therapeutic compositions and methods of the invention. Alternate means for cyclizing the peptides are also within the scope of the invention. As noted above, in various embodiments of the invention one or more amino acid(s) of a compstatin analog (e.g., any of the compstatin analogs disclosed herein) can be an N-alkyl amino acid (e.g., an N-methyl amino acid). For example, and without limitation, at least one amino acid within the cyclic portion of the peptide, at least one amino acid N-terminal to the cyclic portion, and/or at least one amino acid C-terminal to the cyclic portion may be an N-alkyl amino acid, e.g., an N-methyl amino acid. In some embodiments of the invention, for example, a compstatin analog comprises an N-methyl glycine, e.g., at the position corresponding to position 8 of compstatin and/or at the position corresponding to position 13 of compstatin. In some embodiments, one or more of the compstatin analogs in Table 1 contains at least one N-methyl glycine, e.g., at the position corresponding to position 8 of compstatin and/or at the position corresponding to position 13 of compstatin.

TABLE 1

| | | SEQ ID | Activity over |
|---|---|---|---|
| Peptide | Sequence | NO: | compstatin |
| Compstatin | H-ICVVQDWGHHRCT-CONH2 | 8 | * |
| Ac-compstatin | Ac-ICVVQDWGHHRCT-CONH2 | 9 | 3xmore |
| Ac-V4Y/H9A | Ac-ICVYQDWGAHRCT-CONH2 | 10 | 14xmore |
| Ac-V4W/H9A —OH | Ac-ICVWQDWGAHRCT-COOH | 11 | 27xmore |
| Ac-V4W/H9A | Ac-ICVWQDWGAHRCT-CONH2 | 12 | 45xmore |
| Ac-V4W/H9A/T13dT —OH | Ac-ICVWQDWGAHRCdT-COOH | 13 | 55xmore |
| Ac-V4(2-Nal)/H9A | Ac-ICV(2-Nal)QDWGAHRCT-CONH2 | 14 | 99xmore |
| Ac V4(2-Nal)/H9A —OH | Ac-ICV(2-Nal)QDWGAHRCT-COOH | 15 | 38xmore |
| Ac V4(1-Nal)/H9A —OH | Ac-ICV(1-Nal)QDWGAHRCT-COOH | 16 | 30xmore |
| Ac-V42Igl/H9A | Ac-ICV(2-Igl)QDWGAHRCT-CONH2 | 17 | 39xmore |
| Ac-V42Igl/H9A —OH | Ac-ICV(2-Igl)QDWGAHRCT-COOH | 18 | 37xmore |
| Ac-V4Dht/H9A —OH | Ac-ICVDhtQDWGAHRCT-COOH | 19 | 5xmore |
| Ac-V4(Bpa)/H9A —OH | Ac-ICV(Bpa)QDWGAHRCT-COOH | 20 | 49xmore |
| Ac-V4(Bpa)/H9A | Ac-ICV(Bpa)QDWGAHRCT-CONH2 | 21 | 86xmore |
| Ac-V4(Bta)/H9A —OH | Ac-ICV(Bta)QDWGAHRCT-COOH | 22 | 65xmore |
| Ac-V4(Bta)/H9A | Ac-ICV(Bta)QDWGAHRCT-CONH2 | 23 | 64xmore |
| Ac-V4W/H9(2-Abu) | Ac-ICVWQDWG(2-Abu)HRCT-CONH2 | 24 | 64xmore |
| +G/V4W/H9A +AN —OH | H-GICVWQDWGAHRCTAN-CONH | 25 | 38xmore |
| Ac-V4(5fW)/H9A | Ac-ICV(5fW)QDWGAHRCT-CONH₂ | 26 | 31xmore |
| Ac-V4(5-MeW)/H9A | Ac-ICV(5-methyl-W)QDWGAHRCT-CONH₂ | 27 | 67xmore |
| Ac-V4(1-MeW)/H9A | Ac-ICV(1-methyl-W)QDWGAHRCT-CONH₂ | 28 | 264xmore |
| Ac-V4W/W7(5fW)/H9A | Ac-ICVWQD(5fW)GAHRCT-CONH₂ | 29 | 121xmore |
| Ac-V4(5fW)/W7(5fW)/H9A | Ac-ICV(5fW)QD(5fW)GAHRCT-CONH₂ | 30 | 161xmore |
| Ac-V4(5-MeW)/W7(5fW)H9A | Ac-ICV(5-methyl-W)QD(5fW)GAHRCT- | 31 | NA |
| | CONH₂ | | |
| Ac-V4(1MeW)/W7(5fW)/H9A | Ac-ICV(1-methyl-W)QD(5fW)GAHRCT- | 32 | 264xmore |
| | CONH₂ | | |
| +G/V4(6fW)/W7(6fW)H9A+N— | H-GICV(6fW)QD(6fW)GAHRCTN-COOH | 33 | 126xmore |
| OH | | | |
| Ac-V4(1-formyl-W)/H9A | Ac-ICV(1-formyl-W)QDWGAH RCT-CONH₂ | 34 | 264xmore |
| Ac-V4(5-methoxy-W)/H9A | Ac-ICV(5-methoxy-W)QDWGAHRCT-CONH₂ | 35 | 76xmore |
| G/V4(5f-W)/W7(5fW)/H9A+N— | H-GICV(5fW)QD(5fW)GAHRCTN-COOH | 36 | 112xmore |
| OH | | | |

NA = not available

In certain embodiments of the compositions and methods of the invention the compstatin analog has a sequence selected from sequences 9-36. In certain embodiments of the compositions and methods of the invention the compstatin analog has a sequence selected from SEQ ID NOs: 14, 21, 28, 29, 32, 33, 34, and 36. In certain embodiments of the compositions and methods of the invention the compstatin analog has a sequence selected from SEQ ID NOs: 30 and 31. In one embodiment of the compositions and methods of the invention the compstatin analog has a sequence of SEQ ID NO: 28. In one embodiment of the compositions and methods of the invention the compstatin analog has a sequence of SEQ ID NO: 32. In one embodiment of the compositions and methods of the invention the compstatin analog has a sequence of SEQ ID NO: 34. In one embodiment of the compositions and methods of the invention the compstatin analog has a sequence of SEQ ID NO: 29. In one embodiment of the compositions and methods of the invention the compstatin analog has a sequence of SEQ ID NO: 33. In one embodiment of the composition and methods of the invention the compstatin analog has a sequence of SEQ ID NO: 36.

In certain embodiments of the compositions and methods of the invention the compstatin analog has a sequence as set forth in Table 1, but where the Ac- group is replaced by an alternate blocking moiety B¹, as described above. In some embodiments the —NH₂ group is replaced by an alternate blocking moiety B², as described above.

In one embodiment, the compstatin analog binds to substantially the same region of the β chain of human C3 as does compstatin. In one embodiment the compstatin analog is a compound that binds to a fragment of the C-terminal portion of the β chain of human C3 having a molecular weight of about 40 kDa to which compstatin binds (Soulika, A. M., et al., Mol. Immunol., 35:160, 1998; Soulika, A. M., et al., Mol. Immunol. 43(12):2023-9, 2006). In certain embodiments the compstatin analog is a compound that binds to the binding site of compstatin as determined in a compstatin-C3 structure, e.g., a crystal structure or NMR-derived 3D structure. In certain embodiments the compstatin analog is a compound that could substitute for compstatin in a compstatin-C3 structure and would form substantially the same intermolecular contacts with C3 as compstatin. In certain embodiments the compstatin analog is a compound that binds to the binding site of a peptide having a sequence set forth in Table 1, e.g., SEQ ID NO: 14, 21, 28, 29, 32, 33, 34, or 36 in a peptide-C3 structure, e.g., a crystal structure. In certain embodiments the compstatin analog is a compound that binds to the binding site of a peptide having SEQ ID NO: 30 or 31 in a peptide-C3 structure, e.g., a crystal structure. In certain embodiments the compstatin analog is a compound that could substitute for the peptide of SEQ ID NO: 9-36, e.g., a compound that could substitute for the peptide of SEQ ID NO: 14, 21, 28, 29, 32, 33, 34, or 36 in a peptide-C3 structure and would form substantially the same intermolecular contacts with C3 as the peptide. In certain embodiments the compstatin analog is a compound that could substitute for the peptide of SEQ ID NO: 30 or 31 in a peptide-C3 structure and would form substantially the same intermolecular contacts with C3 as the peptide.

One of ordinary skill in the art will readily be able to determine whether a compstatin analog binds to a fragment of the C-terminal portion of the β chain of C3 using routine experimental methods. For example, one of skill in the art could synthesize a photocrosslinkable version of the compstatin analog by including a photo-crosslinking amino acid such as p-benzoyl-L-phenylalanine (Bpa) in the compound, e.g., at the C-terminus of the sequence (Soulika, A. M., et al, supra). Optionally additional amino acids, e.g., an epitope tag such as a FLAG tag or an HA tag could be included to facilitate detection of the compound, e.g., by Western blotting. The compstatin analog is incubated with the fragment and crosslinking is initiated. Colocalization of the compstatin analog and the C3 fragment indicates binding. Surface plasmon resonance may also be used to determine whether a compstatin analog binds to the compstatin binding site on C3 or a fragment thereof and/or to measure binding affinity. A competition experiment, e.g., wherein binding of a compstatin analog to the compstatin binding site on C3 or a fragment thereof interferes with binding of compstatin, may be used. One of skill in the art would be able to use molecular modeling software programs to predict whether a compound would form substantially the same intermolecular contacts with C3 as would compstatin or a peptide having the sequence of any of the peptides in Table 1, e.g., SEQ ID NO: 14, 21, 28, 29, 32, 33, 34, or 36, or in some embodiments SEQ ID NO: 30 or 31.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, WO2004026328, and/or WO2007062249. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3^{rd} ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g. to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

Compstatin or an analog thereof, optionally linked to a binding moiety, can be modified by addition of a molecule such as polyethylene glycol (PEG) or similar molecules to stabilize the compound, reduce its immunogenicity, increase its lifetime in the body, increase or decrease its solubility, and/or increase its resistance to degradation. Methods for pegylation are well known in the art (Veronese, F. M. & Harris, Adv. Drug Deliv. Rev. 54, 453-456, 2002; Davis, F. F., Adv. Drug Deliv. Rev. 54, 457-458, 2002); Hinds, K. D. & Kim, S. W. Adv. Drug Deliv. Rev. 54, 505-530 (2002; Roberts, M. J., Bentley, M. D. & Harris, J. M. Adv. Drug Deliv. Rev. 54, 459-476 (2002; Wang, Y. S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, Calif., which also provides details of appropriate conjugation procedures. In another embodiment compstatin or a compstatin analog is fused to the Fc domain of an immunoglobulin or a portion thereof. In some other embodiments compstatin or a compstatin analog is conjugated to an albumin moiety (e.g., human serum albumin or a portion thereof) or to an albumin binding peptide. Thus in some embodiments compstatin or a compstatin analog is modified with one or more polypeptide or non-polypeptide components, e.g., the compstatin or compstatin analog is pegylated or conjugated to another moiety. In some embodiments the component is not the Fc domain of an immunoglobulin or a portion thereof. Compstatin and/or a compstatin analog can be provided as multimers or as part of a supramolecular complex, which can include either a single molecular species or multiple different species (e.g., multiple different analogs).

In some embodiments, a PEG or other moiety has an average molecular weight of at least 10 kD, e.g., at least 20 kD, 30 kD, 40 kD, 50 kD, 60 kD, 70 kD, 80 kD, 90 kD, 100 kD, 110 kD, 120 kD, 130 kD, 140 kD, 150 kD, or more in various embodiments. For example, an average molecular weight may be between 10 kD and 100 kD, e.g., about 10 kD, 20 kD, 30 kD, 40 kD, 50 kD, 60 kD, 70 kD, 75 kD, 80 kD, 90 kD, or 100 kD.

In some embodiments, a compstatin analog, e.g., modified with PEG or other moiety, has a half-life at least 3, 5, 7, 10, 20, 30, 50, 75, 100-fold or more as great as that of a compstatin analog having the same peptide sequence but lacking the moiety. For example, a half-life may be between 3 and 100-fold as great as that of a compstatin analog having the same peptide sequence but lacking the moiety, e.g., between 3 and 50-fold as great, between 50 and 100-fold as great, etc.

In some embodiments, a compstatin analog, e.g., modified with PEG or other moiety, has a plasma half-life of at least 1, 2, 3, 4, 5, 6, 7, 10, 14, 21, or 28 days. For example, a compstatin analog, e.g., modified with PEG or other moiety, may have a plasma half-life of between 1 day and 28 days, e.g., between 1 day and 4 days, between 4 days and 7 days, etc.

It will be appreciated that a variety of approaches to determining pharmacokinetic (PK) parameters such as half-life can be used. An appropriate method can be selected by one of ordinary skill in the art. In general, half-life can be determined by a method comprising: administering one or more doses of the compound to subjects, obtaining blood samples from the subject at various times after administration, measuring the concentration of the compound in said samples, and calculating a half-life based at least in part on said measurements. For example, in some embodiments, samples may be obtained at times 0 (pre-dose), 5 min, 15 min, 30 min, 1 hr, 4 hr, 8 hr, 24 hr (1 day), 48 hr (2 days), 96 hr (4 days), 192 hr (8 days), 14 days, 21 days, and 28 days post-dose. It will be appreciated that these time points are exemplary. Different time points and/or more or fewer time points could be used in various embodiments. One of ordinary skill in the art would select appropriate time points. The blood samples are typically processed to obtain plasma or serum prior to making the measurements. Any appropriate method for measuring the compound may be used. For example, in some embodiments an immunoassay is used. In some embodiments, a chromatography-based method is used (e.g., liquid chromatography (LC), liquid chromatography-mass spectrometry (LC-MS) or liquid chromatography-tandem mass spectrometry (LC-MS-MS). In some embodiments, a bioassay is used. In many embodiments, the half-life is a terminal (elimination) half-life. In some embodiments, a terminal half-life is calculated following administration of a single dose. In some embodiments, a terminal half-life is calculated following administration of multiple doses and allowing the concentration to reach steady state. In some embodiments, a half-life determined for the initial (distribution) phase is used. For example, if the majority of the compound is removed from circulation during the distribution phase, an initial half-life may be used in some embodiments.

In some embodiments, half-life is determined by conducting a PK analysis using non-compartmental analysis on multiple dose PK data from a group of subjects. In some embodiments, half-life is determined by conducting a PK analysis using a standard 1-compartment model on multiple dose PK data from a group of subjects. In some embodiments, a half-life is determined in subjects who are healthy and not known to be suffering from a disorder. In some embodiments, a half-life is determined in subjects suffering from a complement-mediated disorder. In some embodiments, a half-life is determined in adults (persons at least 18 years of age). A variety of software tools are available to facilitate calculation of PK parameters. For example, Phoenix NMLE or Phoenix WinNonlin software (PharSight Corp, St. Louis, Mo.) or Kinetica (Thermo Scientific) can be used. It will be appreciated that a reasonable estimate of half-life based on a model can be used.

In some embodiments, a multivalent compound comprising a plurality of compstatin analog moieties covalently or noncovalently linked to a polymeric backbone or scaffold is administered to a subject in need of treatment for CRS and/or nasal polyposis. The compstatin analog moieties may be the same or different compstatin analog. The compstatin analog can be any of the compstatin analogs described herein. It will be appreciated that following attachment to the polymeric backbone, the structure of certain compstatin analog moiet(ies) will differ slightly from that of certain of the compstatin analogs described herein. For example, a compstatin analog molecule comprising an amine (NH₂) group, represented as NH₂—R¹, may react with a moiety comprising a carboxylic acid (COOH), represented as R²—(C══O)OH to form a conjugate having formula R²—(C══O)—NH—R¹, in which one of the hydrogens present in the compstatin analog is no longer present and a new covalent bond (C—N) has been formed. Thus the term “compstatin analog moiety” includes molecules having the precise formula of a compstatin analog as described herein as well as molecular structures in which a functional group of a compstatin analog has reacted with a second functional group, which may entail loss of at least one atom or group of atoms that was present in the compstatin analog molecule prior to the reaction and formation of a new covalent bond. The new covalent bond is formed, for example, between an atom that was previously attached to one of the atoms that is lost from the compstatin analog and an atom to which the compstatin analog becomes attached.

The compstatin analog moieties can be identical or different. In certain embodiments of the invention the multivalent compound comprises multiple instances, or copies, of a single compstatin analog moiety. In other embodiments of the invention the multivalent compound comprises one or more instances of each of two of more non-identical compstatin analog moieties, e.g., 3, 4, 5, or more different compstatin analog moieties. In certain embodiments of the invention the number of compstatin analog moieties (“n”) is between 2 and 6. In other embodiments of the invention n is between 7 and 20. In other embodiments of the invention n is between 20 and 100. In other embodiments n is between 100 and 1,000. In other embodiments of the invention n is between 1,000 and 10,000. In other embodiments n is between 10,000 and 50,000. In other embodiments n is between 50,000 and 100,000. In other embodiments n is between 100,000 and 1,000,000.

The compstatin analog moieties may be attached directly to the polymeric scaffold or may be attached via a linking moiety that connects the compstatin analog moiety to the polymeric scaffold. The linking moiety may be attached to a single compstatin analog moiety and to the polymeric scaffold. Alternately, a linking moiety may have multiple compstatin analog moieties joined thereto so that the linking moiety attaches multiple compstatin analog moieties to the polymeric scaffold.

In some embodiments, a compstatin analog comprises an amino acid having a side chain comprising a primary or secondary amine, e.g., a Lys residue. For example, any of the compstatin analog sequences disclosed herein may be extended or modified by addition of a linker comprising one or more amino acids, e.g., one or more amino acids comprising a primary or secondary amine, e.g., in a side chain thereof. For example, a Lys residue, or a sequence comprising a Lys residue, is added at the C-terminus and/or N-terminus of the compstatin analog. In some embodiments, the Lys residue or other amino acid comprising a primary or secondary amine is separated from the cyclic portion of the compstatin analog by a rigid or flexible spacer. A linker or spacer may, for example, comprise a substituted or unsubstituted, saturated or unsaturated alkyl chain, oligo(ethylene glycol) chain, and/or other moieties. The length of the chain may be, e.g., between 2 and 20 carbon atoms. In other embodiments the spacer is or comprises a peptide. The peptide spacer may be, e.g., between 1 and 20 amino acids in length, e.g., between 4 and 20 amino acids in length. Suitable spacers can comprise or consist of multiple Gly residues, Ser residues, or both, for example. Optionally, the amino acid having a side chain comprising a primary or secondary amine and/or at least one amino acid in a spacer is a D-amino acid. A PEG moiety or similar molecule or polymeric scaffold may be linked to the primary or secondary amine, optionally via a linker. In some embodiments, a bifunctional linker is used. A bifunctional linker may comprise two reactive functional groups, which may be the same or different in various embodiments. In various embodiments, one or more linkers, spacers, and/or techniques of conjugation described in Hermanson, supra, is used

Any of a variety of polymeric backbones or scaffolds could be used. For example, the polymeric backbone or scaffold may be a polyamide, polysaccharide, polyanhydride, polyacrylamide, polymethacrylate, polypeptide, polyethylene oxide, or dendrimer. Suitable methods and polymeric backbones are described, e.g., in WO98/46270 (PCT/US98/07171) or WO98/47002 (PCT/US98/06963). In one embodiment, the polymeric backbone or scaffold comprises multiple reactive functional groups, such as carboxylic acids, anhydride, or succinimide groups. The polymeric backbone or scaffold is reacted with the compstatin analogs. In one embodiment, the compstatin analog comprises any of a number of different reactive functional groups, such as carboxylic acids, anhydride, or succinimide groups, which are reacted with appropriate groups on the polymeric backbone. Alternately, monomeric units that could be joined to one another to form a polymeric backbone or scaffold are first reacted with the compstatin analogs and the resulting monomers are polymerized. In another embodiment, short chains are prepolymerized, functionalized, and then a mixture of short chains of different composition are assembled into longer polymers.

### Compstatin Mimetics

The structure of compstatin is known in the art, and NMR structures for a number of compstatin analogs having higher activity than compstatin are also known (Malik, supra). Structural information may be used to design compstatin mimetics.

In some embodiments, the compstatin mimetic is any compound that competes with compstatin or any compstatin analog (e.g., a compstatin analog whose sequence is set forth in Table 1) for binding to C3 or a fragment thereof (such as a 40 kD fragment of the β chain to which compstatin binds). In some embodiments, the compstatin mimetic has an activity equal to or greater than that of compstatin. In some embodiments, the compstatin mimetic is more stable, orally available, or has a better bioavailability than compstatin. The compstatin mimetic may be a peptide, nucleic acid, or small molecule. In certain embodiments the compstatin mimetic is a compound that binds to the binding site of compstatin as determined in a compstatin-C3 structure, e.g., a crystal structure or a 3-D structure derived from NMR experiments. In certain embodiments the compstatin mimetic is a compound that could substitute for compstatin in a compstatin-C3 structure and would form substantially the same intermolecular contacts with C3 as compstatin. In certain embodiments the compstatin mimetic is a compound that binds to the binding site of a peptide having a sequence set forth in Table 1, e.g., SEQ ID NO: 14, 21, 28, 29, 32, 33, 34, or 36, or in certain embodiments SEQ ID NO: 30 or 31, in a peptide-C3 structure. In certain embodiments the compstatin mimetic is a compound that could substitute for a peptide having a sequence set forth in Table 1, e.g., SEQ ID NO: 14, 21, 28, 29, 32, 33, 34, or 36, or in certain embodiments SEQ ID NO: 30 or 31, in a peptide-C3 structure and would form substantially the same intermolecular contacts with C3 as the peptide. In certain embodiments the compstatin mimetic has a non-peptide backbone but has side chains arranged in a sequence designed based on the sequence of compstatin.

One of skill in the art will appreciate that once a particular desired conformation of a short peptide has been ascertained, methods for designing a peptide or peptidomimetic to fit that conformation are well known. See, e.g., G. R. Marshall (1993), Tetrahedron, 49: 3547-3558; Hruby and Nikiforovich (1991), in Molecular Conformation and Biological Interactions, P. Balaram & S. Ramasehan, eds., Indian Acad. of Sci., Bangalore, PP. 429-455), Eguchi M, Kahn M., Mini Rev Med Chem., 2(5):447-62, 2002. Of particular relevance to the present invention, the design of peptide analogs may be further refined by considering the contribution of various side chains of amino acid residues, e.g., for the effect of functional groups or for steric considerations as described in the art for compstatin and analogs thereof, among others.

It will be appreciated by those of skill in the art that a peptide mimic may serve equally well as a peptide for the purpose of providing the specific backbone conformation and side chain functionalities required for binding to C3 and inhibiting complement activation. Accordingly, it is contemplated as being within the scope of the present invention to produce and utilize C3-binding, complement-inhibiting compounds through the use of either naturally-occurring amino acids, amino acid derivatives, analogs or non-amino acid molecules capable of being joined to form the appropriate backbone conformation. A non-peptide analog, or an analog comprising peptide and non-peptide components, is sometimes referred to herein as a “peptidomimetic” or “isosteric mimetic,” to designate substitutions or derivations of a peptide that possesses much the same backbone conformational features and/or other functionalities, so as to be sufficiently similar to the exemplified peptides to inhibit complement activation. More generally, a compstatin mimetic is any compound that would position pharmacophores similarly to their positioning in compstatin, even if the backbone differs.

The use of peptidomimetics for the development of high-affinity peptide analogs is well known in the art. Assuming rotational constraints similar to those of amino acid residues within a peptide, analogs comprising non-amino acid moieties may be analyzed, and their conformational motifs verified, by means of the Ramachandran plot (Hruby & Nikiforovich 1991), among other known techniques.

One of skill in the art will readily be able to establish suitable screening assays to identify additional compstatin mimetics and to select those having desired inhibitory activities. For example, compstatin or an analog thereof could be labeled (e.g., with a radioactive or fluorescent label) and contacted with C3 in the presence of different concentrations of a test compound. The ability of the test compound to diminish binding of the compstatin analog to C3 is evaluated. A test compound that significantly diminishes binding of the compstatin analog to C3 is a candidate compstatin mimetic. For example, a test compound that diminishes steady-state concentration of a compstatin analog-C3 complex, or that diminishes the rate of formation of a compstatin analog-C3 complex by at least 25%, or by at least 50%, is a candidate compstatin mimetic. One of skill in the art will recognize that a number of variations of this screening assay may be employed. Compounds to be screened include natural products, libraries of aptamers, phage display libraries, compound libraries synthesized using combinatorial chemistry, etc. The invention encompasses synthesizing a combinatorial library of compounds based upon the core sequence described above and screening the library to identify compstatin mimetics. Any of these methods could also be used to identify new compstatin analogs having higher inhibitory activity than compstatin analogs tested thus far.

Other Complement Inhibitors

While compstatin analogs are of particular interest herein, the invention relates in some aspects to use of other compounds that inhibit one or more complement pathways or activities for treating CRS and/or nasal polyposis. Such compounds may be used individually (instead of a compstatin analog) or in combination with a compstatin analog in certain embodiments of the invention. Thus where the instant application refers to a compstatin analog, it should be understood that the invention provides an embodiment in which a different complement inhibitor, e.g., any complement inhibitor discussed herein, is used.

Complement inhibitors of use in various embodiments of the invention fall into a number of compound classes such as peptides, polypeptides, antibodies, small molecules (e.g., organic compounds having a molecular weight of 1,500 Daltons (Da) or less, e.g., 1,000 Da or less, e.g., 500 Da or less, and having multiple carbon-carbon bonds) and nucleic acids (e.g., aptamers, RNAi agents such as short interfering RNAs). Complement inhibitors include antagonists of one or more proteins in the classical, alternative, and/or lectin pathway. In certain embodiments of the invention the complement inhibitor inhibits an enzymatic activity of a complement protein. The enzymatic activity may be proteolytic activity, such as ability to cleave another complement protein. In certain embodiments of the invention the complement inhibitor inhibits cleavage of C3, C5, or factor B. In some embodiments, the compound is an antagonist of a C3a receptor (C3aR) or C5a receptor (C5aR).

In certain embodiments, a complement inhibitor inhibits activation of C5. For example, the complement inhibitor may bind to C5 and inhibit its cleavage. In some embodiments, the complement inhibitor inhibits physical interaction of C5 with C5 convertase by, e.g., binding to C5 or C5 convertase or to C5 at a site that would ordinarily participate in such physical interaction. Exemplary agents that inhibit C5 activation include antibodies, antibody fragments, polypeptides, small molecules, and aptamers. Exemplary compounds, e.g., antibodies, that bind to C5 are described, for example, in U.S. Pat. No. 6,534,058; PCT/US95/05688 (WO 1995/029697), PCT/EP2010/007197 (WO2011063980); U.S. Pat. Pub. No. 20050090448; and U.S. Pat. Pub. No. 20060115476. U.S. Pat. Pub. No. 20060105980 discloses aptamers that bind to and inhibit C5. In some embodiments, a humanized anti-C5 monoclonal antibody, e.g., eculizumab (also known as h5G1.1-mAb; Soliris®) (Alexion), or a fragment or derivative thereof that binds to C5. In some embodiments, an antibody comprising at least some of the same complementarity determining regions (CDR1, CDR2 and/or CDR3), e.g., all of CDR1, CDR2, and CDR3, as those of eculizumab's heavy chain and/or light chain is used. In some embodiments, the antibody comprises at least some of the same framework regions as eculizumab. In some embodiments, an antibody that binds to substantially the same binding site on C5 as eculizumab is used. In some embodiments, pexelizumab (also known as h5G1.1-scFv), a humanized, recombinant, single-chain antibody derived from h5G1.1-mAb, is used. In certain embodiments the complement inhibitor comprises a Staphylococcus SSL7 protein from Staphylococcus aureus or a variant or derivative or mimetic of such protein that can bind to C5 and inhibit its cleavage.

Bispecific or multispecific antibodies can be used. For example, PCT/US2010/039448 (WO/2010/151526) discloses bispecific antibodies described as binding to two or more different proteins, wherein at least two of the proteins are selected from C5a, C5b, a cellular receptor for C5a (e.g., C5aR1 or C5L2), the C5b-9 complex, and a component or intermediate of terminal complement such as C5b-6, C5b-7, or C5b-8. In some embodiments an RNAi agent that inhibits expression of C5 or C5aR may be used.

In some embodiments, a complement inhibitor known as OmCI, or a variant, derivative, or mimetic thereof, is used. OmCI binds to C5 and inhibits its activation most likely by inhibiting interaction with convertase. OmCI is naturally produced by the tick Ornithodoros moubata. See, e.g., PCT/GB2004/002341 (WO/2004/106369) and PCT/GB2010/000213 (WO/2010/100396), for description of OmCI and certain variants thereof. It has been shown that OmCI binds to eicosanoids, in particular leukotriene (LKs), e.g., LTB4. In some embodiments, an OmCI polypeptide (or a variant, derivative, or fragment thereof) that retains the capacity to binds to a LK, e.g., LTB4, is used. In some embodiments, an OmCI polypeptide (or a variant, derivative, or fragment thereof) that has reduced capacity or substantially lacks capacity to bind to a LK, e.g., LTB4, is used.

U.S. Pat. No. 6,676,943 discloses human complement C3-degrading protein from Streptococcus pneumoniae. PCT/US2008/001662 (WO/2008/140637) discloses compounds comprising peptides and peptide analogs capable of binding the C3 protein and inhibiting complement activation.

In other embodiments the agent is an antagonist of a C5a receptor (C5aR). Exemplary C5a receptor antagonists include a variety of small cyclic peptides such as those described in U.S. Pat. No. 6,821,950; U.S. Ser. No. 11/375,587; and/or PCT/US06/08960 (WO2006/099330). In certain embodiments of the invention a cyclic peptide comprising the sequence [OPdChaWR] (SEQ ID NO: 37) is used. In certain embodiments of the invention a cyclic peptide comprising the sequence [KPdChaWR] (SEQ ID NO: 38) is used. In certain embodiments a peptide comprising the sequence (Xaa)ₙ[OPdChaWR] (SEQ ID NO: 39) is used, wherein Xaa is an amino acid residue and n is between 1 and 5. In certain embodiments a peptide comprising the sequence (Xaa)ₙ[KPdChaWR] (SEQ ID NO: 40) is used, wherein Xaa is an amino acid residue and n is between 1 and 5. In certain embodiments of the invention n is 1. In certain embodiments of the invention n is 1 and Xaa is a standard or nonstandard aromatic amino acid. For example, the peptides F-[OPdChaWR] (SEQ ID NO: 41), F-[KPdChaWR] (SEQ ID NO: 42); Cin-[OPdChaWR] (SEQ ID NO: 43), and HCin-[OPdChaWR] (SEQ ID NO: 44) are of interest. Optionally the free terminus comprises a blocking moiety, e.g., the terminal amino acid is acetylated. (Abbreviations: O: ornithine; Cha: cyclohexylalanine; Cin: cinnamoyl; Hcin: hydrocinnamoyl; square brackets denote internal peptide bond). Other C5aR antagonists are disclosed in PCT/EP2006/005141 (WO 2006/128670) PCT/EP2006/000365 (WO 2006/074964), and/or PCT/EP2004/008057 (WO 2005/010030).

In certain embodiments of the invention the complement inhibitor is a virus complement control protein (VCCP), such as a poxvirus complement control protein. For example, the complement inhibitor may be vaccinia virus complement control protein (VCP), smallpox inhibitor of complement enzymes (SPICE), or other VCCP described in U.S. Ser. No. 11/247,886 and/or PCT/US2005/36547, filed Oct. 8, 2005. In other embodiments, the complement inhibitor is a complement inhibitor derived from a tick or other haematophagous arthropod. See, e.g., PCT/GB2004/002341 (WO 2004/106369).

In some embodiments of the invention the complement inhibitor is or comprises a naturally occurring mammalian complement regulatory protein or portion thereof. For example, the complement regulatory protein may be CR1, DAF, MCP, CFH, or CFI or may be a chimeric polypeptide comprising portions of two or more complement regulatory proteins. In some embodiments of the invention the complement regulatory polypeptide is one that is normally membrane-bound in its naturally occurring state. In some embodiments of the invention a fragment of such polypeptide that lacks some or all of a transmembrane and/or intracellular domain is used. Soluble forms of complement receptor 1 (sCR1), for example, are of use in certain embodiments of the invention. For example the compounds known as TP10 or TP20 (Avant Therapeutics) can be used. C1 inhibitor (C1-INH) is also of use. In some embodiments a soluble complement control protein, e.g., CFH, is used. In some embodiments of the invention the polypeptide is modified to increase its solubility.

Inhibitors of C1s are of use in certain embodiments of the invention. For example, U.S. Pat. No. 6,515,002 describes compounds (furanyl and thienyl amidines, heterocyclic amidines, and guanidines) that inhibit C1s. U.S. Pat. Nos. 6,515,002 and 7,138,530 describe heterocyclic amidines that inhibit C1s. U.S. Pat. No. 7,049,282 describes peptides that inhibit classical pathway activation. Certain of the peptides comprise or consist of WESNGQPENN (SEQ ID NO: 45) or KTISKAKGQPREPQVYT (SEQ ID NO: 46) or a peptide having significant sequence identity and/or three-dimensional structural similarity thereto. In some embodiments these peptides are identical or substantially identical to a portion of an IgG or IgM molecule. U.S. Pat. No. 7,041,796 discloses C3b/C4b Complement Receptor-like molecules and uses thereof to inhibit complement activation. U.S. Pat. No. 6,998,468 discloses anti-C2/C2a inhibitors of complement activation.

It will be appreciated that variants or fragments of a polypeptide described above that inhibits complement may be used in certain embodiments of the invention. In some embodiments, a variant or fragment of a first polypeptide comprises a polypeptide at least 70%, 75%, 80%, 85%, 90%, 95%, 96%, 97%, 98%, 99%, or more identical to the first polypeptide over at least 70%, 75%, 80%, 85%, 90%, 95%, 96%, 97%, 98%, 99%, or 100% of the first polypeptide. Percent identity may be determined using methods known in the art. For example, computer programs such as BLAST2, BLASTN, BLASTP, Gapped BLAST, etc., generate alignments and provide percent identity between a sequence of interest and sequences in any of a variety of public databases. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. See the Web site having URL www.ncbi.nlm.nih.gov for these programs. In a specific embodiment, percent identity of a sequence of interest and a second sequence is calculated using BLAST2 with default parameters. In some embodiments at least some amino acids are conservatively replaced relative to the reference sequence. Conservative replacements may be defined in accordance with Stryer, L., Biochemistry, 3rd ed., 1988, according to which amino acids in the following groups possess similar features with respect to side chain properties such as charge, hydrophobicity, aromaticity, etc. (1) Aliphatic side chains: G, A, V, L, I; (2) Aromatic side chains: F, Y, W; (3) Sulfur-containing side chains: C, M; (4) Aliphatic hydroxyl side chains: S, T; (5) Basic side chains: K, R, H; (6) Acidic amino acids: D, E, N, Q; (7) Cyclic aliphatic side chain: P, which may be considered to fall within group (1). In another accepted classification, conservative substitutions occur within the following groups: (1) Non-polar: A, L, I, V, G, P, F, W, M; (2) Polar: S, T, C, Y, N, Q. (3) Basic: K, R, H; (4) Acidic: D, E Amino acids with a small side chain (G, A, S, T, M) also form a group from among which conservative substitutions can be made. Other classification methods known in the art can be used. Furthermore, amino acid analogs and unnatural amino acids can be classified in accordance with these schemes.

In some embodiments, a variant or fragment exhibits at least 70%, 75%, 80%, 85%, 90%, 95%, 96%, 97%, 98%, 99%, or more of the complement inhibiting activity of the polypeptide of which it is a variant. In some embodiments, a variant has higher complement inhibiting activity than the polypeptide of which it is a variant. For example, variants of VCP that have greatly enhanced complement inhibiting activity are known in the art. Examples of suitable assays for measuring complement activity are mentioned above.

In some embodiments, a complement inhibitor that binds to substantially the same binding site (e.g., a binding site on a complement component such as C3, C5, factor B, factor D, or an active complement split product) as a complement inhibitor described herein is used. In general, the ability of first and second agents to bind to substantially the same site on a target molecule, such as a complement component or receptor, can be assessed using methods known in the art, such as competition assays, molecular modeling, etc. (See, e.g., discussion of compstatin analog mimetics.) Optionally the first and/or second agent can be labeled with a detectable label, e.g., a radiolabel, fluorescent label, etc. Optionally the target molecule, first agent, or second agent is immobilized on a support, e.g., a slide, filter, chip, beads, etc. In some embodiments, a second antibody that binds to substantially the same binding site as a first antibody comprises one or more CDR(s) that are at least 90%, 91%, 92%, 93%, 94%, 95%, 96%, 97%, 98%, 99%, or 100% identical to CDR(s) of the first antibody.

Pharmaceutical Compositions and Administration

Compstatin analogs may be administered in substantially pure form for treatment of CRS and/or NP, e.g., in a pharmaceutical composition. Suitable preparations, e.g., substantially pure preparations of a compstatin analog, optionally together with one of more additional active agent(s) may be combined with one or more pharmaceutically acceptable carriers or vehicles etc., to produce an appropriate pharmaceutical composition. The term “pharmaceutically acceptable carrier or vehicle” refers to a non-toxic carrier or vehicle that does not destroy the pharmacological activity of the compound with which it is formulated. One of skill in the art will understand that a carrier or vehicle is “non-toxic” if it is compatible with administration to a subject in an amount appropriate to deliver the compound without causing undue toxicity. Pharmaceutically acceptable carriers or vehicles that may be used in the compositions and/or methods of this invention include, but are not limited to, water, physiological saline, Ringer's solution, sodium acetate or potassium acetate solution, 5% dextrose, and the like. The composition may include other components as appropriate for the formulation desired, e.g., as discussed below. Such components are typically compatible with administration to a desired location of the body without producing unacceptable side effects or toxicity. One or more substances that are independently useful for treating a subject suffering from CRS and/or nasal polyposis can be present in the compositions.

Often, a compstatin analog is administered intranasally for treatment of CRS and/or nasal polyposis. For nasal administration, a compstatin analog may be formulated, e.g., as a solution, suspension, gel, dry powder, or microparticle or nanoparticle formulation, in various embodiments. Compositions comprising a compstatin analog in in a dissolved state may be prepared as a solution by any suitable method.

In some embodiments, a solution or suspension is an aqueous composition. In some embodiments, an aqueous composition comprises at least 50% water by volume, e.g., at least 60%, 70%, 80%, 90%, 95%, or more water by volume. Optionally the composition contains one or more co-solvents. Exemplary co-solvents include organic solvents such as ethanol, propylene glycol, and polyethylene glycol (e.g., PEG 300, PEG 400), N—N dimethylacetamide (DMA), N-methyl-2-pyrolidone (NMP), glycerol, and combinations thereof. In some embodiments, a composition may be at least in part lipid based, such as an emulsion, microemulsion, or micellar solution. Lipids in a composition can include, e.g., medium and/or long chain triglycerides, which may be provided in the form of a variety of pharmaceutically acceptable oils, as known in the art.

Solutions can be prepared by incorporating the compound, e.g., compstatin analog, in the required amount in an appropriate solvent, optionally with one or a combination of ingredients such as buffers such as acetates, citrates, lactates or phosphates; agents for the adjustment of tonicity such as sodium chloride or dextrose; antimicrobial agents such as. benzyl alcohol or methyl parabens, propylparaben, butylparaben, chlorobutanol, phenethyl alcohol, phenyl mercuric acetate and benzalkonium chloride; antioxidants such as ascorbic acid, glutathione, or sodium bisulfite; chelating agents such as ethylenediaminetetraacetic acid; and other suitable ingredients etc., as desired, followed by filter-based sterilization.

One of skill in the art will be aware of numerous physiologically acceptable compounds that may be included in a pharmaceutical composition. Other useful compounds include, for example, carbohydrates, such as glucose, sucrose, lactose; dextrans; amino acids such as glycine; polyols such as mannitol, cyclodextrins, etc. These compounds may, for example, serve as bulking agents and/or stabilizers, e.g., in a powder and/or when part of the manufacture or storage process involves lyophilization. Surfactant(s) such as Tween-80, Pluronic-F108/F68, SPANs, deoxycholic acid, phosphatidylcholine, etc., may be included in a composition, e.g., to increase solubility or to provide microemulsion to deliver hydrophobic drugs. pH can be adjusted with acids or bases, such as hydrochloric acid or sodium hydroxide, if desired. Scent or flavoring agents can be included.

A composition can comprise a polymer or other material that may modify one or more properties of the composition, e.g., physical properties such as flow characteristics. Useful materials include, without limitation thereto, sodium carboxy methyl cellulose, alginate, carageenans, carbomers, galactomannans, hydroxypropyl methylcellulose, hydroxypropyl cellulose, polyethylene glycols, polyvinyl alcohol, polyvinylpyrrolidone, sodium carboxymethyl chitin, sodium carboxymethyl dextran, sodium carboxymethyl starch and xanthan gum. In some embodiments, a composition contains microcrystalline cellulose and/or an alkali metal carboxyalkylcellulose. Combinations of any two or more of these materials are also useful in certain embodiments.

Generally, dispersions are prepared by incorporating the active compound into a sterile vehicle which contains a basic dispersion medium and appropriate other ingredients from those enumerated above. In the case of sterile powders for the preparation of sterile injectable solutions, methods of preparation can include vacuum drying and freeze-drying which yields a powder of the active ingredient plus any additional desired ingredient, e.g., from a previously sterile-filtered solution thereof. In some embodiments, a compstatin analog is incorporated into or provided as a component of a microparticle, nanoparticle, liposome, or other drug delivery vehicle, formulation, or device that provides sustained delivery (also referred to as sustained release), protects the compstatin analog from degradation, and/or reduces clearance of the compstatin analog from the nasal cavity or sinuses. In some embodiments, delivery occurs either continuously or intermittently over a period of time e.g., at least 2-7 days, 1, 2, 4, or 6 weeks, at least 1, 2, 3, 4, 6, 8, 10, 12, 15, 18, or 24 months, or longer, e.g., in amounts sufficient to provide a benefit to the subject over such time period.

Nasal solutions can be provided in the form of nasal drops or nasal sprays. A volume of about 25 μl to 200 μl, e.g., about 100 μl can be delivered to either or both nostrils. In some embodiments, the concentration of compstatin analog is between 1 mg/ml and 2000 mg/ml, e.g., between 50 mg/ml and 1000 mg/ml, or between 100 mg/ml and 500 mg/ml. In some embodiments, the concentration administered is between 20 mg/ml and 100 mg/ml. A variety of devices can be used to administer a nasal spray. Suitable devices are known in the art and include, e.g., squeeze bottles, pump sprays, and airless sprays. In some embodiments, a nasal spray contains a compstatin analog dissolved or suspended in a solution or mixture of excipients in a nonpressurized dispenser that delivers a spray containing a metered dose of the compstatin analog. The dose can be metered by the spray pump or may have been premetered during manufacture. In some embodiments, a nasal spray device is designed to be capable of discharging up to several hundred metered sprays of formulation containing the compstatin analog. In some embodiments, a nasal spray device or dry powder delivery device is designed for unit dosing. In some embodiments, the device is disposable, e.g., it contains a single dose (or two doses, one to each nostril) and is not designed to be refilled.

Mechanical pumps or actuators are often employed to deliver nasal formulations as sprays. A variety of devices are available. In some embodiments, a nasal spray is delivered using a Becton-Dickinson Accuspray™ Nasal Delivery System or similar technology. It creates a spray by forcing liquid through a pressure swirl atomizer when the user depresses the plunger on the device. A thin intact sheet of liquid is formed in the shape of a cone at the exit orifice, and breaks up into droplets of an appropriate size for delivery of drugs to the nasal mucosa. A variety of nasal administration systems are available from the Pharma Division of Erich Pfeiffer GmbH (now Aptar Pharma). Available systems include ones suitable for administering liquids and others suitable for powders. In some embodiments, a nasal spray delivery device with the capability to prevent the entrance of microorganisms is used. For example, pumps may employ sterile filtration in conjunction with a venting system in order to prevent microorganisms from entering. Another common approach involves a mechanical tip seal that closes off the orifice at all times except during spraying of the formulation. In some embodiments an airless spray is used, which prevents entry of air into the dispensing device after use. Such approaches may be of particular use if a composition does not contain an antimicrobial agent. Pressurised metered dose inhalers can also be used, e.g., containing a hydrofluoroalkane as a propellant.

In some embodiments, a compstatin analog is administered intranasally using a nebulizer. A nebulizer device may produce a dispersion of droplets in a gas streams by various methods. Jet nebulizers can, for example, use a compressed air or other compressed gas supply to draw liquid up a tube and through an orifice and introduce it into a flowing gas stream as droplets suspended therein, after which the fluid is caused to impact one or more stationary baffles to remove excessively large droplets. Ultrasonic nebulizers use an electrically driven transducer to subject a fluid to high-frequency oscillations, producing a cloud of droplets which can be entrained in a moving gas stream. Hand-held nebulizers may atomize a liquid with a squeeze bulb air supply. A variety of nebulizers are available, e.g., from PARI Respiratory Equipment, Inc. For example, PARI SinuStar™ Nasal Aerosol Delivery System delivers aerosols to the upper airway including the sinuses. The PARI Sinus Therapy System is described as combining efficient nebulization with a vibrating pulse to efficiently deliver aerosol into the paranasal sinuses. The SinusAero™ Nasal Nebulizer (Sinus Dynamics) is another nebulizer system of use.

In some embodiments, controlled particle dispersion technology (CPD) is used. CPD employs the principle of vertical flow, by which inherent airflows of the nasal cavity are disrupted. CPD allows delivery of formulations to the entire nasal cavity, olfactory region, and paranasal sinuses. See, e.g., PCT/US2004/029001 (WO/2005/023335). For example, ViaNase ID (Kurve Technology, Bothell, Wash.) is a CPD-powered electronic atomizer that can be used to deliver a compstatin analog for treatment of CRS and/or nasal polyposis.

In some embodiments, an approach that utilizes the exhalation breath of a user as the driving force to deliver a metered dose of a liquid substance is employed. For example, bidirectional intranasal drug delivery can be used, which delivers a drug while the subject exhales and is reported to reduce lung deposition. It uses the concept that exhalation against resistance leads to closure of the soft palate, thus separating the nasal cavity from the mouth and cutting off communication between the cranial surface of the soft palate and the posterior margin of the nasal septum. Under such conditions, air can enter through one nostril through the sealing nozzle, turn ˜180 degrees, and exit through the other nostril in the reverse direction. A single-use or multidose liquid reservoir or powder delivery device can be used. For example, OptiNose (Oslo, Norway) has developed devices embodying this approach. See, e.g., Djupesland P G, Breath actuated device improves delivery to target sites beyond the nasal valve. Laryngoscope, 116(3):466-72, 2006. See also PCT/IB2007/004353 (WO/2008/081326).

In some embodiments, a DirectHaler Nasal device is used (Direct-Haler, Copenhagen, Denmark, now owned by Trimel BioPharma, Etobicoke, Ontario). In this device, the subject blows air out of the mouth and into the device, upon which a nasal dry powder dose is delivered into the nostril. See, e.g., Keldmann, T., Advanced Simplification of Nasal Delivery Technology: Anatomy+Innovative Device=Added Value Opportunity. ONdrugdelivery, 3^{rd} issue, pp. 4-7 (2005).

In some embodiments, a device or delivery method is selected such that a significant fraction of the administered material is deposited in the nasal cavity posterior to the nasal vestibule. In some embodiments a significant fraction of the administered material is deposited in the region lined with respiratory epithelium (a ciliated pseudo-stratified columnar epithelium). In some embodiments, a significant fraction of the administered material is deposited in the region above the inferior meatus encompassing the middle turbinate, the middle meatus, the sinus ostia of the maxillary, frontal and ethmoidal sinuses, and the olfactory region. In some embodiments, a significant fraction is at least 20%, at least 30%, at least 40%, or at least 50% of particles or dose of compstatin analog. Parameters such as the spray-cone angle, configuration of the delivery device, particle size range, tap density, etc. can be selected to direct the composition to a desired location, e.g., to achieve a more posterior deposition, increase delivery to the middle meatus or sinus ostia, etc. In some embodiments, a delivery device includes a nosepiece which is inserted into the one nostril of a subject and a nozzle through which a substance is delivered to the nasal cavity. Particles having a desired range or distribution of aerodynamic and/or physical particle sizes (e.g., diameters) can be used. In some embodiments, the range or average of a relevant parameter (e.g., aerodynamic particle size) is between 3 μM and 150 μM, e.g., between 5 μM and 100 μM, e.g., between 10-50 μm. In some embodiments, a size range is selected to reduce the likelihood that an inhaled particle would reach or be retained in the lung and/or to reduce the likelihood that a particle would be exhaled. In some embodiments, a desired particle size is at least about 10 μM. In some embodiments, at least 50%, e.g., between 50% and 90% of the particles, or between 90% and 99.99%, can fall within a desired size range and/or fall outside an undesired size range. Methods for assessing the size and physical properties of particles and/or for modeling and assessing the location of particle deposition are known in the art and include, e.g., those described in the United States Pharmacopoeia and/or European Pharmacopoeia.

In some embodiments, a compstatin analog is administered to the nose or to one or more paranasal sinuses as a depot or in a composition that forms a depot upon administration. In some embodiments, the depot forms upon contact with nasal or sinus secretions. In certain embodiments the depot decreases in size and/or density over time (e.g., by degradation and/or disintegration), releasing the compstatin analog. The depot may be in the form of a gel or a material having physical properties (e.g., viscosity, elasticity, hardness, and/or compressibility) characteristic of a gel, wherein a “gel” may be defined as a colloidal system in which a porous network of interconnected particles (typically of nanometer scale) spans the volume of a liquid medium. In some embodiments, the composition comprises a compstatin analog and an excipient that modulates the rate of deposit degradation/disintegration and/or modulates a physical characteristic of the depot. In some embodiments, a composition comprises a bioadhesive, mucoadhesive, and/or viscosity-modifying substance. In some embodiments, the substance reduces the clearance of the compstatin analog in the nasal cavity or sinuses. For example, the composition may adhere to the nasal or sinus mucosa. In some embodiments, the composition comprises a compstatin analog and a gel-forming substance. In some embodiments, the composition comprises a compstatin analog and an excipient that modulates the rate of degradation/disintegration and/or modulates a physical characteristic of the depot. In some embodiments, the excipient is a sugar alcohol or amino acid.

In some embodiments, a compstatin analog is administered as a gel or ointment. A gel, ointment, or other pharmaceutical composition of the invention can contain one or more thickening agents, soothing substances, humectants, or emollients such as glycerin, aloe, propylene glycol, etc.

In some embodiments, a composition contains a substance that enhances absorption through mucus and/or into nasal or sinus mucosa.

In some embodiments, a compstatin analog is administered as a dry powder. The dry powder can be produced using standard technology, which can include spray drying, milling or grinding and optionally seiving a lyophilized preparation of compound, supercritical fluid or dense gas processes, or other suitable methods to obtain particles, e.g., of a desired size range. A dry powder can be composed of solid or hollow particles in various embodiments. The dry powder can contain, e.g., bulking agents, stabilizers, surfactants, buffer substances, or other excipients in addition to the compstatin analog.

In some embodiments, a compstatin analog is delivered to the nasal cavity and/or paranasal sinuses by use of an implant. Often, an implant comprises a polymeric material. In some embodiments, an implant is biodegradable, e.g., by way of diffusion or by degradation of the matrix. Such degradation may release the compstatin analog. An implant, e.g., a biodegradable implant, could have any of a variety of shapes, e.g., rods, pellets, beads, strips, or microparticles, and may be delivered into a sinus in various pharmaceutically acceptable carriers. Such implants may be designed to have a size, shape, density, viscosity, and/or mucoadhesiveness that prevents them from being substantially cleared by the mucociliary lining of the sinuses during a desired treatment period. See, e.g., PCT/US2004/007828 (WO/2004/082525) for a description of certain biodegradable implants and devices and methods for their deployment. In some instances, an instrument for visualizing the sinus ostium or sinus wall is used. Examples of such instruments include endoscopes and computed tomography (CT) scanners.

Exemplary synthetic polymers which can be used to form a biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. In some embodiments, a bioadhesive polymer is used.

It will be understood that the pharmaceutically acceptable compounds and preparation methods mentioned herein are exemplary and non-limiting. See, e.g., Remington: The Science and Practice of Pharmacy. 21st Edition. Philadelphia, Pa. Lippincott Williams & Wilkins, 2005, for additional discussion of pharmaceutically acceptable compounds and methods of preparing pharmaceutical compositions of various types.

It will be appreciated that the compstatin analog and/or additional active agent(s) can be provided as a pharmaceutically acceptable salt. Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Also, pharmaceutically-acceptable salts can be prepared as alkaline metal or alkaline earth salts, such as sodium, potassium or calcium salts, if appropriate depending on the identity of the active agent.

Treatment with a compstatin analog can continue for varying periods of time following the initial administration. It will be appreciated that a variety of different dosing regimens could be used to administer a desired total daily amount. For example, a desired amount of compstatin analog could be administered once, twice, or more during a 24 hour period. In some embodiments, treatment is less frequent, e.g., about every other day or at longer intervals, e.g., if a sustained delivery formulation or device or comptstatin analog comprising a moiety such as PEG that increases the compound's lifetime in the body is used. In some embodiments, administration at intervals ranging, on average, from about 3 days to about 4 weeks is contemplated. Treatment can be on a symptomatic basis, e.g., during exacerbations, or on a chronic basis. Often, treatment is continued for at least about 1, 2, 4, 6 or more weeks, e.g., several months, years, or more. Treatment can be continued indefinitely. Nasal administration could be to a single nostril or to both nostrils depending, e.g., on whether the symptoms are unilateral or bilateral.

A pharmaceutical composition can be administered in an amount effective to achieve a desired therapeutic effect. In some embodiments the amount is demonstrated to achieve such effect in a clinical trial. In some embodiments, an effective amount results in reduced complement activation within nasal cavity and/or sinus tissues. For example, an effective amount may result in reduced deposition of complement activation products. In some embodiments, an effective amount results in reduced presence of immune system cells (e.g., eosinophils) within nasal cavity and/or sinus tissues. In some embodiments, an effective amount reduces deposition of complement activation products and/or number of immune system cells by at least 25%, at least 50%, at least 75%, at least 90%, or more. In some embodiments, the amount of compstatin analog administered per dose or per day (or released per day in the case of a formulation or device that provides sustained release) is between 0.01 mg and 10,000 mg, e.g., between 0.1 mg and 1,000 mg, e.g., between 1 mg and 500 mg, or between 0.001 mg/kg and 100 mg/kg. The amount may be selected based on a variety of factors such as, e.g., the nature and severity of the condition, the particular formulation, response to therapy, age and/or physical characteristics of the subject, etc.

A variety of assessment instruments known in the art can be used to evaluate the severity and/or response to treatment of CRS and/or nasal polyposis, e.g., any of the assessment instruments mentioned above. For example, assessment can use a visual analog scale, CSS, RSDI, SNOT-20, SNOT-22, Lund-Mackay CT score scoring system, scoring system of Lund and Mackay, Lildholdt's Scale, SF-36 (or other QOL assessment instrument).

In some embodiments, an effective amount results in a better average outcome, e.g., at 1, 3, 6, 9, or 12 months, in a group of subjects treated with a compstatin analog as compared with a group of control subjects. Control subjects can be subjects who did not receive a compstatin analog and had or would be expected to have CRS and/or nasal polyps of comparable average severity, as would typically be expected in a randomized trial. The groups of subjects may receive similar care except that the control group is not treated with a compstatin analog. Optionally the control subjects receive a placebo. In some embodiments, an improved outcome, e.g., a reduction in symptoms of CRS and/or nasal polyposis as determined based on at least one clinical assessment instrument is statistically significant. In some embodiments, an effective amount results both in reduced size and/or number of nasal polyps upon imaging and/or nasal examination and in improved quality of life. In some embodiments, an improvement in quality of life is clinically significant.

Subgroups of subjects most likely to experience significant benefit from a compstatin analog, can be identified in clinical trials, if desired. Such subgroups may be defined, e.g., based on severity of the CRS and/or nasal polyposis as determined by one or more criteria prior to treatment, etc.

While local administration to the nasal cavity and/or paranasal sinus(es), e.g., by intranasal administration, is a convenient route of administering a compstatin analog to treat CRS and/or nasal polyposis, it is noted that any appropriate route can be used. For example, a compstatin analog can be administered by the intravenous, subcutaneous, pulmonary, intramuscular, or oral route in various embodiments of the invention. See, e.g., PCT/US2006/039397. In some embodiments, for example, a compstatin analog, e.g., a compstatin analog comprising a moiety such as PEG that increases the compound's lifetime in the body, is administered intravenously. It will be understood that “administration” encompasses directly administering a compound or composition to a subject, instructing a third party to administer a compound or composition to a subject, prescribing or suggesting a compound or composition to a subject (e.g., for self-administration), self-administration, and, as appropriate, other means of making a compound or composition available to a subject.

As noted above, where the instant application refers to a compstatin analog, it should be understood that the invention provides embodiments in which a different complement inhibitor is used. Accordingly, it will be understood that the invention provides embodiments in which any of the pharmaceutical compositions, methods, administration routes, combination therapies, etc., described herein comprises or uses a different complement inhibitor, e.g., any complement inhibitor discussed herein. One of ordinary skill in the art will appreciate that appropriate doses of such agents can be selected, e.g., as described above.

Additional Therapy

The invention encompasses administration of a compstatin analog in combination with additional therapy for CRS and/or nasal polyposis. Such additional therapy may include, in certain embodiments, any appropriate therapy for the condition known in the art. Additional therapy can include administration of one or more therapeutic agents such as a corticosteroid, leukotriene antagonist, anti-IgE agent, anti-histamine, decongestant, beta-agonist, or anti-infective agent.

Examples of anti-infective agents include antibacterial agents, antifungal agents, antiviral agents, and antiseptics. Examples of antibacterial agents include aminoglycosides, amphenicols, ansamycins, lactams, lincosamides, macrolides, nitrofurans, quinolones, sulfonamides, sulfones, tetracyclines, and any of their derivatives. Exemplary antifungal agents include polyenes, allylamines, azoles (e.g., imidazoles, triazoles, and thiazoles), and echinocandins. Exemplary compounds include amphotericin B, nystatin, miconazole, or ketoconazole.

Exemplary corticosteroids include 21-acetoxypregnenolone, alclometasone, algestone, amcinonide, beclomethasone, betamethasone, budesonide, chloroprednisone, ciclesonide, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, rofleponide palmitate, tixocortol, triamcinolone, (e.g., triamcinolone acetonide, triamcinolone benetonide, triamcinolone hexacetonide). In some embodiments, a corticosteroid is selected from mometasone furoate, fluticasone propionate, fluticasone furoate, rofleponide palmitate, budesonide, triamcinolone acetonide, prednisolone, beclomethasone dipropionate, ciclesonide, and flunisolide.

Exemplary decongestants include 1-desoxyephedrine, ephedrine, ephedrine hydrochloride, ephedrine sulfate, naphazoline, naphazoline hydrochloride, oxymetazoline and pharmaceutically acceptable salts thereof, oxymetazoline hydrochloride, phenylephrine, phenylpropanolamine, menazoline, phenylephrine hydrochloride, propylhexedrine, xylometazoline and xylometazoline hydrochloride.

Exemplary anti-histamines include anti-histamine, such as azelastine, loratidine, brompheniramine, chlorpheniramine, mizolastine, promethazine, doxylamine, desloratidine, triprolidine, clemastine, fexofenadine, cetirizine and levocetirizine, and the pharmaceutically-acceptable salts and derivatives thereof.

As used herein, the term “leukotriene antagonist” encompasses leukotriene receptor antagonists (e.g., zafirlukast) and leukotriene synthesis inhibitors (e.g., zileuton).

An “anti-IgE agent” is an agent that inhibits or antagonizes IgE, e.g., by binding to IgE and blocking its interaction with its receptor(s). Exemplary anti-IgE agents include antibodies (e.g., omalizumab) or IgE receptor antagonists.

One of skill in the art can select an appropriate amount and/or dose of other agent(s) described herein that may be used in conjunction with a compstatin analog. For example, e.g., in some embodiments an amount or dose ranging from about 0.001 mg/kg to 1,000 mg/kg body weight, e.g., about 0.01 to 25 mg/kg body weight, e.g., about 0.1 to 20 mg/kg body weight, e.g., about 1 to 10 mg/kg of a compound described herein is administered at various intervals and over different periods of time as appropriate. The skilled artisan will appreciate that certain factors can influence the total amount, dosage(s) and timing required to effectively treat a subject, including but not limited to the severity of the condition, other treatments being administered, the general health and/or age of the subject, and diseases that may be present. In any embodiment, a conventional dose of the second agent can be used.

When two or more therapies (e.g., compounds or compositions) are used or administered “in combination” with each other, they may be given at the same time, within overlapping time periods, or sequentially (e.g., separated by up to 2 weeks in time), in various embodiments of the invention. They may be administered via the same route or different routes. In some embodiments, at least the compstatin analog is administered intranasally. In some embodiments, the compounds or compositions are administered within 48 hours of each other. In some embodiments, a compstatin analog can be given prior to or after administration of the additional compound(s), e.g., sufficiently close in time that the compstatin analog and additional compound(s) are present at useful levels within the body at least once. In some embodiments, the compounds or compositions are administered sufficiently close together in time such that no more than 90% of the earlier administered composition has been metabolized to inactive metabolites or eliminated, e.g., excreted, from the body, at the time the second compound or composition is administered.

In some embodiments, a composition that includes both the compstatin analog and additional compound(s) is administered. For example, a composition (e.g., a nasal spray or other composition for intranasal administration) may contain a compstatin analog and a corticosteroid or leukotriene antagonist. In some aspects, the invention provides a composition comprising a compstatin analog and a second agent useful for treating CRS and/or nasal polyposis wherein the composition is suitable for intranasal administration. In some embodiments, the second agent is a corticosteroid, leukotriene antagonist, anti IgE agent, or antiinfective agent. In some embodiments, the composition is provided as a nasal spray. In some aspects, the invention provides a nasal administration device, e.g., any of the devices described above or a device implementing any of the nasal delivery approaches discussed above, wherein the device contains any of the inventive compositions.

### Example 1

Human subjects between the ages of 18 and 85 diagnosed with chronic rhinosinusitis and mild to moderate bilateral nasal polyposis are enrolled in a clinical study. Subjects are randomly assigned to treatment and placebo groups. A compstatin analog (e.g., compstatin analog of SEQ ID NO: 28) in a liquid composition is administered twice daily by nasal spray to subjects in the treatment group. The second group receives placebo. Change in polyp size is measured using Lildholdt's Scale. The percentage of subjects showing a reduction in polyp size equal to or greater than 1 on the Lildholdt's Scale when compared to placebo at 4, 8, and 12 weeks is determined. The summed polyp score at 4, 8, and 12 weeks is compared between groups. A reduction in average summed polyp score and/or a greater number of subjects showing a decrease in summed polyp score in the treatment group as compared with the placebo group is indicative of efficacy. The average change in peak nasal inspiratory inflow at 12 weeks is compared between groups. The number of subjects experiencing a clinically significant increase in peak inspiratory inflow at 12 weeks is also compared between the groups. A greater average increase in peak nasal inspiratory inflow and/or a greater percentage of subjects experiencing a clinically meaningful increase in peak nasal inspiratory inflow at 12 weeks in the treatment group as compared with the placebo group is indicative of efficacy.

### Example 2

Human subjects between the ages of 18 and 85 diagnosed with chronic rhinosinusitis and moderate to severe nasal polyposis in one or both nostrils and without a history of CRS are enrolled in a clinical study. Subjects are randomly assigned to treatment and placebo groups. A compstatin analog (e.g., compstatin analog of SEQ ID NO: 28) in a liquid composition is administered twice daily by nasal spray to subjects in the treatment group. The second group receives placebo. Change in polyp size is measured using Lildholdt's Scale. The percentage of subjects showing a reduction in polyp size equal to or greater than 1 on the Lildholdt's Scale when compared to placebo at 4, 8, and 12 weeks is determined The summed polyp score at 4, 8, and 12 weeks is compared between groups. A reduction in average summed polyp score and/or a greater number of subjects showing a decrease in summed polyp score in the treatment group as compared with the placebo group is indicative of efficacy. The average change in peak nasal inspiratory inflow at 12 weeks is compared between groups. The number of subjects experiencing a clinically significant increase in peak inspiratory inflow at 12 weeks is also compared between the groups. A greater average increase in peak nasal inspiratory inflow and/or a greater percentage of subjects experiencing a clinically meaningful increase in peak nasal inspiratory inflow at 12 weeks in the treatment group as compared with the placebo group is indicative of efficacy.

### Example 3

A study as described in Example 1 is performed with subjects that have NP do not have a history or diagnosis of CRS.

### Example 4

A study as described in Example 2 is performed with subjects that have NP do not have a history or diagnosis of CRS.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. It will be appreciated that the invention is in no way dependent upon particular results achieved in any specific example or with any specific embodiment. In the claims articles such as “a”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. For example, and without limitation, it is understood that where claims or description indicate that a residue at a particular position may be selected from a particular group of amino acids or amino acid analogs, the invention includes individual embodiments in which the residue at that position is any of the listed amino acids or amino acid analogs. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more elements, limitations, clauses, or descriptive terms, found in any other claim that is dependent on the same base claim. Furthermore, where the claims recite a composition, it is to be understood that methods of administering the composition according to any of the methods disclosed herein, and methods of using the composition for any of the purposes disclosed herein are included within the scope of the invention, and methods of making the composition according to any of the methods of making disclosed herein are included within the scope of the invention, unless otherwise indicated or unless it would be evident to one of ordinary skill in the art that a contradiction or inconsistency would arise. Methods of treating a subject can include a step of providing a subject in need of such treatment, e.g., a subject who has or is at increased risk of having CRS and/or nasal polyposis, a step of diagnosing a subject as having CRS and/or nasal polyposis or as having a condition associated with increased risk of CRS and/or nasal polyposis, and/or a step of selecting a subject for treatment with a compstatin analog.

Where elements are presented as lists, e.g., in Markush group format, it is to be understood that each subgroup of the elements is also disclosed, and any element(s) can be removed from the group. For purposes of conciseness only some of these embodiments may have been specifically recited herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also to be understood, any particular embodiment, aspect, element, feature, etc., of the present invention may be explicitly excluded from the claims whether or not such exclusion is not set forth explicitly herein.

1. A method of treating a subject in need of treatment for chronic rhinosinusitis (CRS) or nasal polyposis, the method comprising administering a complement inhibitor to the subject.

2. The method of claim 1, wherein the subject has CRS with nasal polyposis.

3. (canceled)

4. The method of claim 1, wherein the subject has asthma, NSAID sensitivity, and CRS with nasal polyposis.

5. The method of claim 1, wherein the subject has asthma, NSAID sensitivity, and nasal polyposis.

6. The method of claim 1, wherein the subject has nasal polyps but does not have CRS.

7.-9. (canceled)

10. The method of claim 1, wherein the complement inhibitor is administered intranasally.

11. (canceled)

12. The method of claim 1, wherein the complement inhibitor is a compstatin analog.

13. (canceled)

14. The method of claim 1, wherein the complement inhibitor is a compstatin analog, and wherein the compstatin analog is a compound that comprises a cyclic peptide having a core sequence of X′aa-Gln-Asp-Xaa-Gly (SEQ ID NO: 3), where X′aa and Xaa are selected from Trp and analogs of Trp.

15. The method of claim 1, wherein the complement inhibitor is a compstatin analog, and wherein the compstatin analog is a compound that comprises a cyclic peptide having a core sequence of X′aa-Gln-Asp-Xaa-Gly-X″aa (SEQ ID NO: 4), where X′aa and Xaa are each independently selected from Trp and analogs of Trp and X″aa is selected from His, Ala, single methyl unbranched amino acids, Phe, Trp, and analogs of Trp.

16. (canceled)

17. The method of claim 1, wherein the complement inhibitor is a compstatin analog, and wherein the compstatin analog is a compound that comprises a cyclic peptide having a sequence of X′aa1-X′aa2-X′aa3-X′aa4-Gln-Asp-Xaa-Gly-X″aa1-X″aa2-X″aa3-X″aa4-X″aa5 (SEQ ID NO: 5), where X′aa4 and Xaa are selected from Trp and analogs of Trp, wherein X′aa1, X′aa2, X′aa3, X″aa1, X″aa2, X″aa3, X″aa4, and X″aa5 are independently selected from among amino acids and amino acid analogs, and the peptide is cyclized via a bond between X′aa2 and X″aa4.

18. (canceled)

19. The method of claim 1, wherein the complement inhibitor is a compstatin analog, and wherein the compstatin analog is a compound that comprises a cyclic peptide having a sequence:
Xaa1-Cys-Val-Xaa2-Gln-Asp-Xaa2*-Gly-Xaa3-His-Arg-Cys-Xaa4 (SEQ ID NO: 6); wherein:
Xaa1 is Ile, Val, Leu, B¹-Ile, B¹-Val, B¹-Leu or a dipeptide comprising Gly-Ile or B¹-Gly-Ile, and B¹ represents a first blocking moiety;
Xaa2 and Xaa2* are independently selected from Trp and analogs of Trp;
Xaa3 is His, Ala or an analog of Ala, Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of the L-Thr, D-Thr, Ile, Val, Gly, Ala, or Asn optionally is replaced by a second blocking moiety B²; and
the two Cys residues are joined by a disulfide bond.

20. The method of claim 19, wherein
Xaa1 is Ile, Val, Leu, Ac-Ile, Ac-Val, Ac-Leu or a dipeptide comprising Gly-Ile or Ac-Gly-Ile;
Xaa2 and Xaa2* are independently selected from Trp and analogs of Trp;
Xaa3 is His, Ala or an analog of Ala, Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of the L-Thr, D-Thr, Ile, Val, Gly, Ala, or Asn optionally is replaced by —NH₂.

21. The method of claim 19, wherein Xaa2 is an analog of Trp having increased hydrophobic character relative to Trp.

22. The method of claim 19, wherein Xaa2 is an analog of Trp comprising a substituted or unsubstituted bicyclic aromatic ring component or two or more substituted or unsubstituted monocyclic aromatic ring components.

23. The method of claim 19, wherein Xaa2* is an analog of Trp having an electronegative substituent on the indole ring and not having increased hydrophobic character relative to Trp.

24. The method of claim 19, wherein the compstatin analog has a sequence selected from the group consisting of: SEQ ID NOs: 9-36.

25. (canceled)

26. The method of claim 19, wherein the compstatin analog has the sequence of SEQ ID NO: 28, 32, or 34.

27. The method of claim 1, wherein the complement inhibitor is a compstatin analog, and wherein the compstatin analog is a compound that comprises a cyclic peptide having a sequence of X′aa1-X′aa2-X′aa3-X′aa4-Gln-Asp-Xaa-Gly-X″aa1-X″aa2-X″aa3-X″aa4-X″aa5 (SEQ ID NO: 5), where X′aa4 and Xaa are selected from Trp and analogs of Trp, wherein X′aa1, X′aa2, X′aa3, X″aa1, X″aa2, X″aa3, X″aa4, and X″aa5 are independently selected from among amino acids and amino acid analogs, X′aa2 and X″aa4 are not Cys, and the peptide is cyclized via a bond between X′aa2 and X″aa4.

28. (canceled)

29. The method of claim 27, wherein the bond is an amide bond, wherein one of X′aa2 and X″aa4 is an amino acid or amino acid analog having a side chain that comprises a primary or secondary amine, the other one of X′aa2 and X″aa4 is an amino acid or amino acid analog having a side chain that comprises a carboxylic acid group, and the bond is an amide bond.

30. The method of claim 15, wherein the peptide is acetylated at the N-terminus, amidated at the C-terminus, or both acetylated at the N-terminus and amidated at the C-terminus.

31.-34. (canceled)

35. A composition comprising a complement inhibitor and a second compound useful for treating CRS or nasal polyposis.

36.-39. (canceled)
